(19) United States
(12) Patent Application Publication
(10) Pub. No.: US 2023/0384313 A1
(43) Pub. Date: Nov. 30, 2023

(54) METHODS FOR DIAGNOSING GASTRIC INTESTINAL METAPLASIA

(71) Applicant: ARIZONA BOARD OF REGENTS ON BEHALF OF ARIZONA STATE UNIVERSITY, Scottsdale, AZ (US)

(72) Inventors: Joshua LaBaer, Chandler, AZ (US); Ji Qiu, Chandler, AZ (US); Lusheng Song, Tempe, AZ (US); Yunro Chung, Chandler, AZ (US)

(21) Appl. No.: 18/322,442

(22) Filed: May 23, 2023

Related U.S. Application Data
(60) Provisional application No. 63/345,209, filed on May 24, 2022.

(51) Int. Cl. G01N 33/574

(52) U.S. Cl. CPC G01N 33/57446; G01N 2333/205; G01N 2800/52; G01N 2800/50

(57) ABSTRACT

Provided are methods and compositions for identifying individuals having intestinal metaplasia (IM) and/or Helicobacter pylori infection based on the levels of antibodies against particular Helicobacter pylori antigens. Patient diagnosis allows for medical interventions, including therapeutic treatments, disease progression preventative measures and enhanced cancer screening. Also provided are methods and compositions for identifying individuals having IM who are at increased risk for developing gastric cancer. Patient diagnosis and stratification by risk allows for targeted screening and monitoring for signs of gastric cancer as well as earlier intervention with cancer preventative treatments.

### CROSS-REFERENCE TO RELATED APPLICATIONS

This application claims priority to, and the benefit of, U.S. Application No. 63/345,209 filed May 24, 2022, the content of which is incorporated herein by reference in its entirety.

### STATEMENT REGARDING FEDERALLY SPONSORED RESEARCH

This invention was made with government support under RO1 CA199948 awarded by the National Institutes of Health. The government has certain rights in the invention.

### REFERENCE TO AN ELECTRONIC SEQUENCE LISTING

The contents of the electronic sequence listing (112624.01399.xml; Size: 22,690 bytes; and Date of Creation: May 23, 2023) is herein incorporated by reference in its entirety.

### BACKGROUND

Gastric cancer (GC) is a major public health burden, representing the third leading cause of cancer mortality in the world. Helicobacter pylori (H. pylori) chronic infection is the primary causative factor attributed to about 90% of noncardia GC. H. pylori-driven gastric carcinogenesis is a multistep process with well-defined histological stages, variably progressing from chronic non-atrophic gastritis (NAG) to atrophic gastritis with or without gastric intestinal metaplasia (IM), dysplasia, and cancer.

Gastric IM is a heterogeneous precancerous lesion with variable prevalence across populations. The estimated overall annual risk of gastric cancer in patients with IM in endoscopy-based studies is 0.34%. International guidelines recommend endoscopic surveillance of patients with advanced IM as defined by histological features (i.e., incomplete type) and anatomical location (i.e., extension to the corpus).

There is a need in the art for methods for diagnosing IM and identifying subjects in need of treatment for IM and prioritized screening for gastric cancer. There also is a need in the art for methods with improved discriminatory power for assessing risks of developing gastric cancer and identifying subjects in need of heightened screening and monitoring for signs of gastric cancer. In addition, there is a need for non-invasive tests to diagnose and identify these high-risk individuals, especially in resource-limited settings.

### SUMMARY

Disclosed herein are compositions and methods for diagnosing intestinal metaplasia (IM) and H. pylori infection.

In an aspect, provided herein is a method comprising: (a) contacting an antibody-containing sample obtained from a subject with at least one H. pylori antigen selected from the group consisting of HP0547/CagA, HP1125/PalA, HP0596/Tipα, HP1177/Omp27, HP0103/TlpB, HP0709, HP0900/HypB, HP0371/FabE, HP0243/NapA, HP0153/RecA, and HP0385 to allow for antigen-antibody binding; and (b) measuring the level of antigen-specific antibodies bound to the at least one antigen. In embodiments, step (a) comprises contacting the antibody-containing sample with at least three of the H. pylori antigens, and step (b) comprises measuring the level of antigen-specific antibodies bound to the at least three antigens. In embodiments, step (a) comprises contacting the antibody-containing sample with at least five of the H. pylori antigens, and step (b) comprises measuring the level of antigen-specific antibodies bound to each of the at least five antigens. In embodiments, the method further comprises: (c) screening the subject for gastric cancer if the level of the antigen-specific antibody bound to the at least one antigen is increased compared to a subject with non-atrophic gastritis, wherein the increased antigen is selected from the group consisting of HP0547/CagA, HP1125/PalA, HP0596/Tipα, HP1177/Omp27, and HP0103/TpB; and/or if the at least one antigen is decreased compared to a subject with non-atrophic gastritis, wherein the decreased antigen is selected from the group consisting of HP0709, HP0900/HypB, HP0371/FabE, HP0243/NapA, HP0153/RecA, and HP0385.

In embodiments, the method further comprises administering to the subject an antibiotic to treat Helicobacter pylori infection if the level of the antigen-specific antibody bound to the at least one antigen is increased compared to a subject with non-atrophic gastritis, wherein the increased antigen is selected from the group consisting of HP0547/CagA, HP1125/PalA, HP0596/Tipα, HP1177/Omp27, and HP0103/TlpB; and/or if the at least one antigen is decreased compared to a subject with non-atrophic gastritis, wherein the decreased antigen is selected from the group consisting of HP0709, HP0900/HypB, HP0371/FabE, HP0243/NapA, HP0153/RecA, and HP0385.

In embodiments, the method further comprises administering a gastric cancer vaccine to the subject if the level of the antigen-specific antibody bound to the at least one antigen is increased compared to a subject with non-atrophic gastritis, wherein the increased antigen is selected from the group consisting of HP0547/CagA, HP1125/PalA, HP0596/Tipα, HP1177/Omp27, and HP0103/TpB; and/or if the at least one antigen is decreased compared to a subject with non-atrophic gastritis, wherein the decreased antigen is selected from the group consisting of HP0709, HP0900/HypB, HP0371/FabE, HP0243/NapA, HP0153/RecA, and HP0385.

In embodiments, the method further comprises before step (b), contacting the antibody bound to the antigen with a detectable binding agent; and step (b) comprises measuring the level of antigen specific antibodies using the detectable binding agent.

In embodiments, the subject is human and the detectable binding agent comprises one or more anti-human IgG and/or anti-human IgA antibodies comprising a detectable label or conjugated to a detectable label. In embodiments, step (b) further comprises immobilizing antibody-antigen complexes to a solid support. In embodiments, the sample comprises at least one of blood, plasma, and serum.

In embodiments, the method further comprises contacting the sample with at least one antigen selected from the group consisting of HP0516/HslU, HP0385, and HP1453/HomD and measuring the level of antigen-specific antibodies in the sample, wherein increased levels of antibodies compared to a subject with corpus extension is indicative of antral restricted intestinal metaplasia.

In another aspect, provided herein is a method comprising: (a) contacting an antibody-containing sample obtained from a subject with at least one H. pylori antigen selected from the group consisting of HP1118/Ggt, HP1110/PorA, HP0870/FlgE, HP0407/BisC, HP0601/FlaA, HP1126/TolB, HP1527/ComH, HP0492, HP1564/PlpA, HP0231, HP0295/Fla, HP1555/Tfs, HP0010/GroEL, HP0709, HP1341/TonB2, and HP0477/HopJ to allow for antigen-antibody binding; and (b) measuring the level of antigen-specific antibodies bound to the at least one antigen.

In embodiments, the method further comprises treating the subject with antibiotics if the level of antigen-specific antibodies bound to the at least one antigen is increased compared to non-infected or past infected controls. In embodiments, the method further comprises before step (b), contacting the antibody bound to the antigen with a detectable binding agent; and wherein step (b) comprises measuring the level of antigen specific antibodies using the detectable binding agent.

In embodiments, the subject is human and the detectable binding agent comprises one or more anti-human IgG and/or anti-human IgA antibodies comprising a detectable label or conjugated to a detectable label. In embodiments, step (b) comprises immobilizing antibody-antigen complexes to a solid support. In embodiments, the sample comprises at least one of blood, plasma, and serum. In embodiments, step (a) comprises contacting the antibody-containing sample with at least five of the H. pylori antigens, and step (b) comprises measuring the level of antigen-specific antibodies bound to each of the at least five antigens.

In an aspect, provided herein is a kit for detecting intestinal metaplasia comprising: (a) at least three H. pylori antigen selected from the group consisting of HP1125/PalA, HP0596/Tipα, HP1177/Omp27, HP0103/TlpB, HP0709, HP0900/HypB, HP0371/FabE, HP0153/RecA, and HP0385; or at least one H. pylori antigen selected from the group consisting of HP1118/Ggt, HP1110/PorA, HP0870/FlgE, HP0407/BisC, HP0601/FlaA, HP1126/TolB, HP1527/ComH, HP0492, HP1564/PlpA, HP0231, HP0295/Fla, HP1555/Tfs, HP0010/GroEL, HP0709, HP1341/TonB2, and HP0477/HopJ; and (b) components for detecting antibodies capable of binding to the antigens.

In an aspect, provided herein is a kit for detecting a current H. pylori infection comprising: (a) at least one H. pylori antigen selected from the group consisting of HP1118/Ggt, HP1110/PorA, HP0870/FlgE, HP0407/BisC, HP0601/FlaA, HP1126/To1B, HP1527/ComH, HP0492, HP1564/P1pA, HP0231, HP0295/Fla, HP1555/Tfs, HP0010/GroEL, HP0709, HP1341/TonB2, and HP0477/HopJ; and (b) components for detecting antibodies capable of binding to at least one of the antigens.

### BRIEF DESCRIPTION OF THE DRAWINGS

The present invention will be better understood and features, aspects, and advantages other than those set forth above will become apparent when consideration is given to the following detailed description thereof. Such detailed description makes reference to the following drawings.

FIG. 1. A proposed model of gastric carcinogenesis cascade despite loss of many anti-H. pylori antibodies. NAG: non-atrophic gastritis, AG: Atrophic gastritis, IM: intestinal metaplasia, DYS: dysplasia.

FIG. 2. Pairwise Pearson correlations (ranged −0.15 to 0.56) among the 11 validated anti-H. pylori IgG and 2 IgA antibodies differential between intestinal metaplasia cases and non-atrophic gastritis controls in the validation sample set. All pairwise comparisons with correlations higher than 0.2 were statistically significant with p-values <0.01.

FIG. 3. Pairwise Pearson correlations (ranged -0.06 to 0.53) among the 15 validated anti-H. pylori IgG and 1 IgA antibodies differential between current and past H. pylori infection in the validation sample set. All pairwise comparisons with correlation higher than 0.20 were statistically significant with p-values <0.01.

FIGS. 4A-4B. Expression and serum analysis quality control. Inter-array Pearson correlations of anti-GST and pooled sample for discovery (A) and validation (B) arrays. Expression accessing and internal positive serum sample reproducibility for discovery (A) and validation (B) sets.

FIGS. 5A-5F. The number of seropositive IgG and IgA proteins in 1528-gene H. pylori NAPPA by H. pylori serology (A, B), histology (C, D), and Urease tests (E, F) in intestinal metaplasia cases (IM, n=50) and non-atrophic gastritis controls (NAG, n=50) in the discovery set. The median and interquartile ranges are presented as lines.

FIGS. 6A-6B. Heatmap of IgG antibodies (62) (A) and IgA antibodies (11) (B) with more than 10% seropositivity on H. pylori-NAPPA. Short and long arrows indicate antibodies that were high in IM cases and low in IM, respectively. The color scale is listed on the right using the NMI value.

FIG. 7. Receiver operating characteristics area under the curve (AUC) for multiple-antibodies (11) panels built with logistic regression model and anti-CagA antibody. The AUC value for the 11 Abs was 0.81 (95% CI,0.75-0.87), for anti-CagA antibody was 0.77 (95% CI, 0.70-0.84).

FIGS. 8A-8B. Heatmap of IgG antibodies (62) (A) and IgA antibodies (11) (B) with more than 10% seropositivity on H. pylori-NAPPA. Arrows indicate antibodies that were high in current infection cases. The color scale is listed on the right using the NMI value.

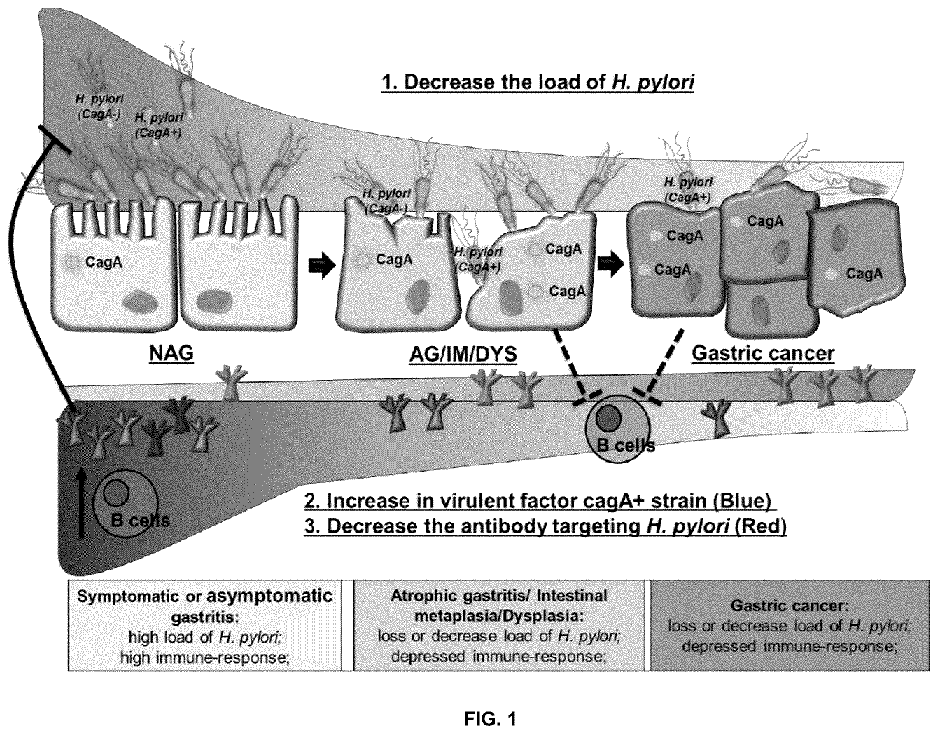

### DETAILED DESCRIPTION

The methods, compositions, kits, and systems for diagnosing intestinal metaplasia (IM) and H. pylori infection provided herein are based at least in part on the inventors' development and validation of panels of antibody biomarkers useful for predicting gastric cancer development. In particular, this disclosure relates to the development and validation of unique H. pylori immunoproteomic profiles useful for identifying subjects having IM and H. pylori infection as well as an increased risk of gastric cancer relative to subject-matched controls. Certain immunoglobulin levels to particular H. pylori proteins or antigens may either positively or negatively correlate with IM, H. pylori infection, and/or increased risk of developing gastric cancer. Further, certain signatures of multiple immunoglobulin levels to particular H. pylori proteins or antigens correlate with risk of developing gastric cancer (GC).

Chronic H. pylori infection is the major risk factor for gastric cancer (GC). However, only some infected individuals develop GC after H. pylori infection. As described herein, methods and compositions have been developed to detect plasma antibodies to H. pylori as biomarkers for an increased risk of gastric cancer. In particular, the methods and compositions uniquely identify subjects having a decreased immune response to H. pylori proteins. Unbound by theory and without being bound to any particular mechanism or mode of action, such subjects may have a reduced ability to mount an immune response against the H. pylori bacterium and, thus, are at greater risk for developing gastric cancer.

Accordingly, in a first aspect, this disclosure provides methods for diagnosing a subject with intestinal metaplasia and/or identifying a subject as having intestinal metaplasia and an increased risk of developing gastric cancer. The method may comprise (a) contacting an antibody-containing sample obtained from a subject with at least on H. pylori antigens selected from the antigens listed in Table 1, specifically HP0547/CagA, HP1125/PalA/Omp18, HP0596/Tipα, HP1177/HopQ/Omp27, HP0103/TlpB, HP0709, HP0900/HypB, HP0371/FabE, HP0243/NapA, HP0153/RecA, and HP0385 to allow for antigen-antibody binding; and (b) measuring the level of antigen-specific antibodies bound to each of the at least one antigens. If the level of antibodies in the sample specific for the at least one antigens is increased compared to a subject with non-atrophic gastritis, when the at least one antigen is selected from HP0547/CagA, HP1125/PalA, HP0596/Tipα, HP1177/Omp27, and HP0103/TlpB, the subject is more likely to have intestinal metaplasia and an increased risk for developing gastric cancer. If the level of antibodies in the sample specific for the at least one antigens is decreased compared to a subject with non-atrophic gastritis, when the at least one antigen is selected from HP0709, HP0900/HypB, HP0371/FabE, HP0243/NapA, HP0153/RecA, and HP0385, the subject is more likely to have intestinal metaplasia and an increased risk for developing gastric cancer. Accordingly, if the subject has a higher likelihood of having intestinal metaplasia and an increased risk for developing gastric cancer, the method may further comprise (c) screening the subject for gastric cancer. The antibodies may comprise three or more of anti-HP0547/CagA, anti-HP1125/PalA, anti-HP0596/Tipα, anti-HP1177/Omp27, anti-HP0103/TlpB, anti-HP0709, anti-HP0900/HypB, anti-HP0371/FabE, anti-HP0243/NapA, anti-HP0153/RecA, and anti-HP0385; or one or more of anti-HP1125/PalA, anti-HP0596/Tipα, anti-HP1177/Omp27, anti-HP0103/TlpB, anti-HP0709, anti-HP0900/HypB, anti-HP0371/FabE, anti-HP0153/RecA, and anti-HP0385. In an embodiment, the antibody comprises an anti-HP0596/Tipα IgA and/or an anti-HP1125/PalA IgA. The method may comprise measuring the level of antigen-specific antibodies to at least 2, 3, 4, 5, 6, 7, 8, 9, 10 or all 11 antigens listed in Table 1.

TABLE 1
Helicobacter Pylori Proteins and Antibody Biomarkers (Reference species H. pylori 26695)

| | | Genbank | | |
|---|---|---|---|---|
| Antigen Name | Description | Accession | Sequence | Antibody |
| HP0103/TlpB | methyl-accepting | NP_206903.1 | SEQ ID NO: 1 | anti- |
| | chemotaxis protein | | | HP0103/TlpB |
| | TlpB | | | |
| HP0153/RecA | recombinase RecA | NP_206952.1 | SEQ ID NO: 2 | anti- |
| | | | | HP0153/RecA |
| HP0243/NapA | DNA protection during | NP_207041.1 | SEQ ID NO: 3 | anti- |
| | starvation protein | | | HP0243/NapA |
| HP0371/FabE | biotin carboxyl carrier | NP_207169.1 | SEQ ID NO: 4 | anti- |
| | protein | | | HP0371/FabE |
| HP0385 | hypothetical protein | NP_207183.1 | SEQ ID NO: 5 | anti-HP0385 |
| HP0547/CagA | CagA | NP_207343.1 | SEQ ID NO: 6 | anti- |
| | | | | HP0547/CagA |
| HP0596/Tipα | tumor necrosis factor | NP_207391.1 | SEQ ID NO: 7 | anti- |
| | alpha-inducing protein | | | HP0596/Tipα |
| HP0709 | S-adenosyl-l- | NP_207503.1 | SEQ ID NO: 8 | anti-HP0709 |
| | methionine hydroxide | | | |
| | adenosyltransferase | | | |
| HP0900/HypB | hydrogenase/urease | NP_207693.1 | SEQ ID NO: 9 | anti- |
| | nickel incorporation | | | HP0900/HypB |
| | protein HypB | | | |
| HP1125/PalA | peptidoglycan | NP_207916.1 | SEQ ID NO: 10 | anti- |
| | associated lipoprotein | | | HP1125/PalA |
| HP1177/Omp27 | membrane protein | NP_207968.1 | SEQ ID NO: 11 | anti- |
| | | | | HP1177/Omp27 |

As used herein, the term intestinal metaplasia or “IM” refers to condition where an epithelium (e.g. an esophagus or stomach epithelium) has become a metaplastic epithelium that closely resembles normal small intestinal epithelium containing goblet cells secreting mucins and sometimes mature, nonsecretory absorptive cells and/or columnar cells in various stages of differentiation secreting mucins (intermediate cells). In some cases, intestinal metaplasia presents with goblet cells that secrete sulfomucins and/or sialomucins. In some cases, intestinal metaplasia presents with a metaplastic epithelium containing acid mucin-producing goblet cells and absorptive enterocytes with a brush border. In other cases, intestinal metaplasia presents without a brush border. In some cases, the intermediate cells secrete mainly sulfomucins. In some cases, the goblet cells secrete sulfomucins and/or sialomucins and the intermediate cells secrete mainly sulfomucins. Non-limiting examples of IM include gastric IM (GIM) and Barrett's esophagus.

As used herein, the term “gastric cancer” (also known as “stomach cancer”) refers to a cancer of the stomach or of stomach cells. Gastric cancer generally develops from neoplastic cells in the lining of the stomach (mucosa or stomach epithelium) and may be in pylorus, body, or cardiac (lower, body and upper) parts of the stomach. Gastric cancer often remains asymptomatic or exhibits only nonspecific symptoms in its early stages. Consequently, diagnosis in many cases is not made until the disease has reached an advanced stage. In some cases, the methods of the disclosure comprise measuring a level of a biomarker associated with gastric cancer in a biological sample obtained from a subject, preferably a combination of antibody biomarkers selected from Table 1.

As used herein, the term “antibody,” or “antibody molecule” (used synonymously herein) refer to naturally occurring immunoglobulin molecule with varying structures. Typically, antibodies are gamma globulin proteins that can be found in blood or other bodily fluids of vertebrates and are used by the immune system to identify foreign materials, such as bacteria, viruses, and toxins. Antibodies bind, by non-covalent interactions, with high affinity to other molecules or structures known as antigens. This binding is specific in the sense that an antibody molecule will only bind to a specific structure with high affinity. The unique part of the antigen recognized by an antibody molecule is called an epitope, or antigenic determinant. The part of the antibody molecule binding to the epitope is sometimes called paratope and resides in the so-called variable domain, or variable region (Fv) of the antibody. The variable domain comprises three complementary-determining regions (CDR's) spaced apart by framework regions (FR's). There are at least five major classes of antibodies: IgA, IgD, IgE, IgG, and IgM, and several of these may be further divided into subclasses (isotypes), e.g. , IgG1, IgG2, IgG3, IgG4, IgA1, and IgA2. They are typically made of basic structural units—each with two large heavy chains and two small light chains—to form, for example, monomers with one unit, dimers with two units or pentamers with five units. Non-limiting examples of antibodies include any known class and/or isotype, such as, e.g., IgA (e.g. IgA1, IgA2, and sIgA), IgD, IgE, IgG (e.g. IgG1, IgG2, IgG3, or IgG4), and IgM. The “class” of an antibody may refer to the type of constant domain or constant region possessed by its heavy chain. For example, heavy chain constant domains may be used to classify different classes of immunoglobulins, such as, e.g., α, γ, δ, ϵ, and μ. The light chain of an antibody may be assigned to certain types, e.g. kappa and lambda, based on the amino acid sequence of its constant domain. In some embodiments, the antibody being measured is IgA or IgG. The subject may be human, and the antibody being measured may be a human antibody.

As used herein, the terms “measure” and “measuring” refer to identifying a quantitative level of the material be measured. As used herein, the term measuring with regard to an antibody or antibody level refers to both detecting and determining a relative and/or absolute amount of an antibody in sample. Standard detection and measuring methods for antibodies include, for example, radioisotope immunoassay, an enzyme-linked immunosorbent assay (ELISA), SISCAPA (Stable Isotope Standards and Capture by Anti-Peptide Antibodies, mass spectrometry, immunofluorescence assays, Western blot, affinity chromatography (e.g. affinity ligand bound to a solid phase), fluorescent antibody assays, immunochromatography, and in situ detection with labeled antibodies. Although any appropriate method can be selected, taking various factors into consideration, ELISA methods are particularly sensitive. In some embodiments, the detecting step or the measuring step comprise an ELISA or Western Blot assay.

The method of diagnosing IM may further comprise screening the subject diagnosed with intestinal metaplasia for gastric cancer, such as, e.g., via endoscopic surveillance or other routine techniques known in the art like barium-meal gastric photofluorography and serum pepsinogen analysis (see e.g. Thrift, A. and El-Serag, H., Clin Gastroenterol Hepatol. (2020) 18:534-542; Kim, B. and Cho, S., Gastrointest Endosc Clin N Am. (2021) 31: 489-501).

The method may further comprise administering to the subject an antibiotic to treat H. pylori infection. The method may further comprise administering a gastric cancer vaccine to the subject diagnosed with intestinal metaplasia.

The method may further comprise before step (b), contacting the antibody bound to the antigen with a detectable binding agent; wherein step (b) comprises measuring the level of antigen specific antibodies using the detectable binding agent. The subject may be human and the detectable binding agent comprises one or more anti-human IgG and/or anti-human IgA antibodies comprising a detectable label or conjugated to a detectable label. The measuring step (b) may comprise immobilizing antibody-antigen complexes to a solid support. The measuring step (b) may comprise an ELISA or Western Blot assay. The sample may comprise a blood, plasma, and/or serum sample.

As used herein, the term “solid support” refers to any substrate or support matrix to which a molecule (such as a lipid, antibody, or antibody-lipid complex) can be bound, either reversibly or irreversibly. Non-limiting examples of solid supports include, without limitation, a bead, plate, slide, flow chamber, chip, cartridge, flow cell, etc. The solid support may be formed from glass, ceramic, polymers (e.g., plastic, latex, polystyrene, polyacrylamide, polyvinylchloride, polypropylene, polyethylene, polylactic acid), cellulose (e.g., paper). In some embodiments, a lipid is immobilized on a solid support. In some embodiments, the lipid is a phospholipid. The lipid may be phosphatidic acid (PA), phosphatidyl choline (PC), and/or phosphatidylserine (PS). In some embodiments, the immobilized lipid is bound to the solid support, either reversibly or irreversibly, to form a platform for an antibody binding reaction. In some embodiments, there are two or more lipids which are immobilized on the solid support in configuration so as to form a lipid panel.

As used herein, the term “bead” refers to a microbead or relatively small bead having a diameter less than about 500 μm, typically with a diameter of less than about 100 μm, 50 μm, or 10 μm. While a bead may vary in shape, typically a bead is substantially spherical, e.g. a microsphere. Microbeads are known in the art and can comprise various materials such as, e.g., latex, polystyrene, polyacrylamide, polyvinylchloride, polypropylene, polyethylene, polylactic acid, ceramic, glass, and magnetic compositions.

As used herein, the term “plate” encompasses a solid support comprising a variety of types, including plastic or glass plates. In some embodiments, the plate is a 96-well, 384-well, or 1536-well plastic plate.

The method may comprise the step of washing away unbound antibodies after contacting the sample with a reagent.

The method may comprise immobilizing antibody-antigen complexes to a solid support. The solid support may comprise a bead, plate, or flow cell.

The method may comprise flowing the sample through a flow-chamber or plurality of microfluidic chambers.

The detecting or measuring step may comprise using a detectable binding agent to determine the presence/absence and/or level of antigen specific antibodies. The detectable binding agent may comprise one or more anti-human IgG and/or anti-human IgA antibodies comprising a detectable label or conjugated to a detectable agent.

As used herein, a “detectable binding agent” refers to a molecule that binds, either directly or indirectly, to a target molecule, and can be detected either directly, or with further chemical or enzymatic reaction. By way of example, but not by way of limitations, a detectable binding agent may comprise an antibody linked to an enzyme, such as horseradish peroxidase (HRP). Addition of an HRP substrate, under enzymatic reaction conditions, will allow detection of the HRP-bound molecule and the target. Other examples of detectable binding agents and include antibodies linked to detectable labels, such as fluorescent labels. Such constructs are well known in the art and are not intended to limit the scope of the present methods and compositions.

As used herein, a “detectable agent” or “detectable label” refers to an agent or label that can be detected either directly, or with further chemical or enzymatic reaction. By way of example, but not by way of limitations, a detectable agent or label may comprise a fluorescent moiety or an enzyme, such as HRP. Other examples of detectable agents and labels include radioisotope. Such agents and labels are well known in the art and are not intended to limit the scope of the present methods and compositions.

The method of diagnosing IM may further comprise contacting the sample with at least one antigen selected from the group consisting of HP0516/Hs1U, HP0385, and HP1453/HomD and measuring the level of antigen-specific antibodies in the sample, wherein increased levels of antibodies compared to a subject with corpus extension is indicative of antral restricted intestinal metaplasia.

The methods disclosed herein comprise measuring the levels of antibodies, for example, IgG or IgA antibodies having specificity for H. pylori proteins. For example, target analytes include IgG or IgA antibodies specifically targeting H. pylori proteins that show a statistically significant difference in intestinal metaplasia (IM) diagnosis. In some cases, therefore, the methods comprise detecting IgG and IgA antibodies having specificity for H. pylori proteins in a sample obtained from a subject. As described and demonstrated in the Examples, reduced seroreactivity for certain antibodies is associated with intestinal metaplasia (IM) and/or an increased risk of gastric cancer relative to a predetermined value or reference sample (e.g., a sample obtained from a subject free of gastric cancer). Such antibodies include some of those listed in Table 1. Also as described and demonstrated in the Examples, increased seroreactivity for certain antibodies is associated with intestinal metaplasia (IM) and/or an increased risk of gastric cancer relative to a predetermined value or reference sample (e.g., a sample obtained from a subject free of gastric cancer). Such antibodies include some of those listed in Table 1. Thus, determining the risk prediction from a multi-antibody signature may comprise both comparing correlations with reduced and increased seroreactivity, depending on the particular antibody biomarker.

As used herein, the term “seroreactivity” refers to a level and/or presence of reactivity to specific antibodies in a sample (e.g., biological sample of a subject or a pooled sample from multiple subjects) as determined using techniques known in the art, such as ELISA. As used herein, the terms “seronegativity” and “seronegative” refer to a reduced level or negative result (or a subject having a negative result) in a test of blood serum, e.g., obtaining a negative result for the presence of an antigen-specific antibody, relative to a control or predetermined value. The term seronegative can encompass subjects for whom blood tests do not reveal the presence of particular antibodies, which can mean the patient does not possess the antibodies, or the patient possesses low levels of the antibodies that cannot be detected by a particular assay. As used herein, the terms “seropositivity” and “seropositive” refer to a positive result (or a subject having a positive result) in a test of blood serum, e.g., obtaining a positive result for the presence of an antigen-specific antibody. The term seropositive can encompass patients for whom blood tests reveal the presence of particular antibodies.

As described in this disclosure, it was determined that increased seroreactivity to particular antigen-specific antibodies relative to a predetermined value or control (e.g., a control sample obtained from a subject that does not have intestinal metaplasia or gastric cancer) is indicative of intestinal metaplasia or an increased risk of developing gastric cancer. The increased seroreactivity relative to a reference may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, at least or about at 100% increase, at least or about at 200% increase, or more.

As described in this disclosure, it was determined that reduced seroreactivity to particular antigen-specific antibodies relative to a predetermined value or control (e.g., a control sample obtained from a subject that does not have intestinal metaplasia or gastric cancer) is indicative of

IM or an increased risk of gastric cancer. It will be understood that absolute seronegativity is not required to determine that a subject has IM or an increased risk of gastric cancer. Instead, reduced seroreactivity for IgG and IgA antibodies having specificity for H. pylori proteins in a sample obtained from a subject may indicate an increased risk of IM or gastric cancer. The decreased level of reactivity may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease. In some cases, the control is a subject that is free of IM or gastric cancer but who exhibits a similar rate of H. pylori infection (−80%) to the test subject. As demonstrated herein, titers of the discovered and validated antibodies in samples obtained from gastric cancer patients were much lower than in samples obtained from persons free of gastric cancer but not necessarily seronegative.

In a second aspect, provided herein is a method of detecting H. pylori infection comprising (a) contacting an antibody-containing sample obtained from a subject with at least one H. pylori antigen selected from the group consisting of HP1118/Ggt, HP1110/PorA, HP0870/FlgE, HP0407/BisC, HP0601/FlaA, HP1126/TolB, HP1527/ComH, HP0492, HP1564/PlpA, HP0231, HP0295/Fla, HP1555/Tfs, HP0010/GroEL, HP0709, HP1341/TonB2, and HP0477/HopJ to allow for antigen-antibody binding; and (b) measuring the level of antigen-specific antibodies bound to the antigen. Increased levels of antibodies as compared to non-infected or past infected controls are indicative of H. pylori infection. The method may further comprise treating a subject having increased levels of the at least one antibody with antibiotics. The method may comprise measuring the level of antigen-specific antibodies to at least 2, 3, 4, 5, 6, 7, 8, 9, 10, 11, 12, 13, 14, 15, or all 16 of the antigens.

As used herein, the term “H. pylori infection” or “Helicobacter pylori infection” refers to an infection of the stomach, sometimes associated with a peptic ulcer or gastritis but which can also be asymptomatic. In some cases, H. pylori infection may present with a sign or symptom such as abdominal pain, nausea, loss of appetite, frequent burping, bloating, and/or unintentional weight loss.

The method of diagnosing H. pylori infection may further comprise before the measuring step (b), contacting the antibody bound to the antigen with a detectable binding agent; wherein step (b) comprises measuring the level of antigen specific antibodies using the detectable binding agent. The subject may be human and the detectable binding agent may comprise one or more anti-human IgG and/or anti-human IgA antibodies comprising a detectable label or conjugated to a detectable label. The measuring step (b) may comprise immobilizing antibody-antigen complexes to a solid support. The measuring step may comprise an ELISA or Western Blot assay. The sample may comprise a blood, plasma, and/or serum sample.

The diagnosing step may be performed prior to any onset of a sign or symptom of IM or H. pylori infection.

The disclosed methods may use a combination of biomarkers that shows a sensitivity and specificity of at least about 85%, at least about 90%, at least about 95%, at least about 98% and about 100%.

In a third aspect, provided herein is a method of determining a multi-antibody signature for two or more antibodies specific to two or more H. pylori antigens, the method comprising detecting or measuring the levels of the two or more antibody types in the sample that specifically bind to immobilized H. pylori antigens. The method can comprise or consist essentially of (a) contacting the sample to a panel of immobilized H. pylori antigens under conditions that promote formation of antigen-antibody complexes; and (b) identifying complexes formed by immobilized H. pylori antigens and antibody in the sample, to determine an H. pylori antibody signature. The antibody signature can be a level of antibody specifically binding to each immobilized antigen. In some cases, the method further comprises comparing an antibody signature from one individual to a predetermined signature and/or to the antibody signature from another individual or patient population. One individual may have a disease, and one individual may be a healthy individual, and the method can allow comparison of the antibody signature in the healthy individual and the individual with a disease. The disease may comprise gastric cancer, intestinal metaplasia or H. pylori infection depending on the antigens used. The immobilized H. pylori antigens may comprise any of the antigens listed in Table 1.

As used herein, the term “multi-antibody signature” refers to a readout comprising data for two or more antibodies in a sample. The readout data may comprise the presence or absence of each antibody type (e.g. one or more antibodies listed in Table 1), the level of each antibody type (e.g. protein level or antigen saturation), and/or the seroreactivity level of each antibody type.

As used herein, the term “panel” refers to a collection of two or more biomolecules (e.g. antigens) wherein the two or more biomolecules are organized such that they can be differentiated from each other in a detecting step. This organization can be a spatial arrangement (e.g. biomolecule 1 is in section 1 of the panel, biomolecule 2 is in section 2 of the panel, etc.). The organization can be a difference in a detectable aspect, e.g. each biomolecule can be associated with a different detectable agent or label (e.g. biomolecule 1 can be detected with color 1, biomolecule 2 can be detected with color 2, etc.).

In a fourth aspect, provided herein are compositions and methods for identifying subjects

having increased risk of developing gastric cancer.

Provided herein is a method comprising or consist essentially of (a) contacting a biological sample obtained from a subject with a reagent composition that comprises components for detecting the presence of two or more antibodies selected from Table 1; and (b) detecting the presence/absence or level of the antibodies in the sample to create a multi-antibody signature, wherein certain multi-antibody signatures are indicative of an increased risk of developing gastric cancer. The detecting step can be used to generate “an antibody multi-signature” which is compared to predetermined antibody signatures to inform a diagnosis or healthcare decision, such as heightened screening for gastric cancer. The predetermined antibody levels can be obtained from a reference sample obtained from an individual or a group of individuals who have gastric cancer and/or later developed gastric cancer.

The method may comprise (a) reacting a biological sample obtained from a subject with a reagent composition that comprises components for determining a level of antibodies to two or more H. pylori antigens listed in Table 1; (b) determining levels of the antibodies in the biological sample; and (c) comparing the levels to predetermined values indicative of gastric cancer, wherein if the levels of two or more antibodies in the biological sample is indicative of an increased risk of developing gastric cancer, then the subject is (i) screened for gastric cancer more frequently than those at lower risk, (ii) given a gastric cancer vaccine, and/or (iii) given a gastric cancer treatment.

The method may comprise (a) contacting an antibody-containing sample, derived from a subject, with an antigen panel configured on a solid support, the antigen panel comprising two or more antigens listed in Table 1; (b) incubating the sample with the antigens to allow for antibody binding; (c) contacting the bound antibodies with a detectable binding agent; (d) detecting the detectable binding agent; (e) measuring the level of antibodies bound to each of the antigens based on the detecting step (d); (f) comparing the measured levels of step (e) or a composite multi-antibody signature to predetermined antibody levels or a predetermined multi-antibody signature. The solid support may comprise a plate, bead, flow chamber, microfluidic chamber, or microchip comprising a plurality of microfluidic chambers.

The method may comprise a step of comparing a multi-antibody signature or various detected antibody levels to at least one predetermined multi-antibody signature for the same antibodies or predetermined antibody levels for the same antibodies.

The method may comprise diagnosing the subject as having an increased risk for developing GC if the multi-antibody signature and/or measured antibody levels is indicative of an increased risk of developing GC. The diagnosing step may be performed prior to any onset of a sign or symptom of GC. The method may comprise performing a preventive measure on the subject if identified as having an increased risk of GC in order to reduce the risk of developing GC and/or to reduce the severity of a potential occurrence of GC. The preventive measure may comprise administering a preventive gastric cancer vaccine to the subject. The method may comprise administering a cancer vaccine to the subject.

The method may comprise screening the subject for GC if identified as having an increased risk of GC. Screening includes performing more frequent and/or numerous tests for signs of GC in the subject as compared to a patient or patient population lacking an indication of increased risk of developing GC.

The method may comprise administering a gastric cancer treatment to the subject if the subject is identified as having GC, e.g. as a result of screening, or identified as having an increased risk of developing GC. In some embodiments, the gastric cancer treatment comprises one or more of vaccine-based therapy, chemotherapy, hormonal therapy, radiotherapy, surgery, and immunotherapy.

The method may comprise administering an effective amount of a treatment regimen to treat gastric cancer. In some cases, the treatment regimen comprises one or more of vaccine-based therapy, chemotherapy, hormonal therapy, radiotherapy, surgery, and immunotherapy.

The antibody-containing sample derived from the subject may be one or more of a whole blood sample, serum sample, and plasma sample. Any suitable blood sample obtained from the subject may be used, including but not limited to whole blood, serum, and blood plasma. In some embodiments, a blood plasma sample is used. Methods for obtaining and preparing blood samples are well known in the art; such methods include those described herein. In one embodiment, plasma is prepared by centrifuging a blood sample under conditions suitable for pelleting of the cellular component of the blood. As demonstrated in the Examples below, certain antibodies show inverse associations with GC, so accordingly, in some embodiments the treatment regimen comprise boosting production of at least one anti-H. pylori antibody, such as e.g., anti-HP0709, anti-HP0900/HypB, anti-HP0371/FabE, anti-HP0243/NapA, anti-HP0153/RecA, and anti-HP0385. For example, the treatment regimen may comprise administering a vaccine-based gastric cancer treatment to the subject, whereby the vaccine results in an increase in the subject's antibody level to one or more H. pylori antigens, preferably an antigen listed in Table 1.

If the measured level is greater than a predetermined antibody value for one or more antibodies, respectively and independently, then, depending on the particular antibodies, the subject may be diagnosed as having IM and/or at an increased risk for developing GC. If the measured level is less than a predetermined antibody value for one or more antibodies, respectively and independently, then, depending on the particular antibodies, the subject may be diagnosed as having IM and/or at increased risk for developing GC. If the measured level is greater than a predetermined antibody value for at least one of the antibodies and is less than another predetermined antibody value for a second antibody, then depending on the particular antibodies, the subject may be diagnosed as having IM and/or at increased risk for developing GC. The level of each antibody in the multi-antibody signature may be evaluated as above, such as e.g. as many as 3, 4, 5, 6, 7, 8, 9, 10 or more antibodies included in a given signature.

### Compositions

In a fifth aspect, disclosed herein are compositions for determining and/or detecting at least two antibody biomarkers associated with gastric cancer or the risk of developing gastric cancer. The reagent composition may be used for producing a multi-antibody signature from a sample derived from a subject.

The composition may comprise components for detecting in a biological sample one or more antibodies listed in Table 1. The antibodies may comprise at least three of: anti-HP0547/CagA, anti-HP1125/PalA/Omp18, anti-HP0596/Tipα, anti-HP1177/HopQ/Omp27, HP0103/TlpB, anti-HP0709, anti-HP0900/HypB, anti-HP0371/FabE/AccB, anti-HP0243/NapA, anti-HP0153/RecA, and anti-HP03859. The antibodies may comprise one or more of anti-HP1125/PalA/Omp18, anti-HP0596/Tipα, anti-HP1177/HopQ/Omp27, anti-HP0103/TlpB, anti-HP0709, anti-HP0900/HypB, anti-HP0371/FabE, anti-HP0153/RecA, and anti-HP0385.

The composition may comprise two or more antigens listed in Table 1. The antigens may be selected from: (i) at least three of HP0547/CagA, HP1125/PalA/Omp18, HP0596/Tipα, HP1177/HopQ/Omp27, HP0103/TlpB, HP0709, HP0900/HypB, HP0371/FabE, HP0243/NapA, HP0153/RecA, and HP0385; or (ii) one or more of HP1125/PalA/Omp18, HP0596/Tipα, HP1177/HopQ/Omp27, HP0103/TlpB, HP0709, HP0900/HypB, HP0371/FabE, HP0153/RecA, and HP0385. The composition may comprise an antigen panel. The composition may be a reagent composition for use in a method described herein. The composition may be lyophilized.

### Articles of Manufacture

In a sixth aspect, disclosed herein are kits for detecting antibody biomarkers associated

with intestinal metaplasia and/or an increased risk of developing gastric cancer. A kit may comprise a reagent composition for producing a multi-antibody signature from a sample derived from a subject, such as a reagent composition described herein.

The kit may comprise a reagent composition that comprises components for detecting in a biological sample the presence of two or more antibody biomarkers of intestinal metaplasia and/or the risk of developing gastric cancer, such as two or more antibodies listed in Table 1. The antibody biomarkers may be selected from at least three of: anti-HP0547/CagA, anti-HP1125/PalA/Omp18, anti-HP0596/Tipα, anti-HP1177/Omp27, HP0103/TlpB, anti-HP0709, anti-HP0900/HypB, anti-HP0371/FabE, anti-HP0243/NapA, anti-HP0153/RecA, and anti-HP0385. In other embodiments, the antibody biomarkers comprise one or more of anti-HP1125/PalA/Omp18, anti-HP0596/Tipα, anti-HP1177/HopQ/Omp27, anti-HP0103/TlpB, anti-HP0709, anti-HP0900/HypB, anti-HP0371/FabE, anti-HP0153/RecA, and anti-HP0385.

The kit may comprise a reagent composition comprising one or more antigens listed in Table 1. In some embodiments, the antibody biomarkers comprise the set of antigens selected from (i) at least three of HP0547/CagA, HP1125/PalA/Omp18, HP0596/Tipα, HP1177/HopQ/Omp27, HP0103/TlpB, HP0709, HP0900/HypB, HP0371/FabE, HP0243/NapA, HP0153/RecA, and HP0385; or (ii) one or more of HP1125/PalA/Omp18, HP0596/Tipα, HP1177/HopQ/Omp27, HP0103/TlB, HP0709, HP0900/HypB, HP0371/FabE, HP0153/RecA, and HP0385. The antigens may be immobilized on a solid support.

The kit may further comprise instructions for performing a method of the disclosure, such as diagnosing gastric cancer or identifying a subject having increased risk of gastric cancer. The kit may further comprise materials for obtaining and preserving a biological sample from a subject. The kit may further comprise a composition described herein and one additional reagent or device, such as a flow chamber, bead composition, ELISA reagent, anti-human IgG antibody, and/or a detectable reagent such as a HRP substrate. The kit may comprise instructions for using the components of the kit in the performance of a method described herein.

### Additional Definitions and Terminology

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

The term “subject” may be used interchangeably with the terms “individual” and “patient” and includes human and non-human subjects. As used herein, the term “subject” refers to one who receives medical care, attention or treatment and may encompass a human patient. As used herein, the term “individual” is meant to encompass a person who has IM or GC, is suspected of having IM or GC, or is at risk of IM or GC. As used herein, “at risk of IM or GC” means that the subject may be asymptomatic or suffering from one or more symptoms of IM or GC such as acid reflux, ulcers, gastritis, GERD, discomfort in the upper abdomen, a feeling of fullness, and the like, but has not been diagnosed with gastric cancer.

The methods for detecting IM of this disclosure can be used as methods for diagnosing gastric cancer and are effective for detecting IM or GC at an early stage and/or prior to the onset of a sign or symptom. As used herein, the term “symptom onset” refers to the time point where the subject presents one or more symptoms characteristic of gastric cancer or IM. Exemplary symptoms of gastric cancer include but are not limited to stomach pain, fatigue, feeling bloated after eating, feeling full after eating small amounts of food, severe persistent heartburn, severe indigestion, unexplained persistent nausea, persistent vomiting, and unintentional weight loss. In another aspect, provided herein is a method for assessing the risk for gastric cancer in a subject, i.e., the likelihood of gastric cancer being present in the subject and/or the likelihood of the subject developing the disease at a later time.

As used herein, the term “treat” or “treatment” encompasses both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The terms “biomolecular marker,” “biomarker,” or “marker” (also sometimes referred to herein as a “target analyte”) are used interchangeably and refer to a molecule whose measurement provides information as to the state of a subject. In various exemplary embodiments, the biomarker is used to assess a pathological state, or the lack thereof. Measurements of the biomarker may be used alone or combined with other data obtained regarding a subject in order to determine the state of the subject. In one embodiment, the biomarker is “differentially present” in a sample taken from a subject of one phenotypic status (e.g., having IM or GC) as compared with another phenotypic status (e.g., not having IM or GC). In one embodiment, the biomarker is “differentially present” in a sample taken from a subject undergoing no therapy or one type of therapy as compared with another type of therapy. Alternatively, the biomarker may be “differentially present” even if there is no phenotypic difference, e.g. the biomarkers may allow the detection of asymptomatic risk. A biomarker may be determined to be “differentially present” in a variety of ways, for example, between different phenotypic statuses if the mean or median level (particularly the expression level of antibodies specific to the H. pylori proteins described herein) of the biomarker in the different groups is calculated to be statistically significant. Common tests for statistical significance include, among others, t-test, ANOVA, Kruskal-Wallis, Wilcoxon, Mann-Whitney and odds ratio.

The actual measurement of levels of a target analyte can be determined (for example, at the protein level) using any method(s) known in the art. A molecule or analyte such as a protein, polypeptide or peptide, or a group of two or more molecules or analytes such as two or more proteins, polypeptides or peptides, is “measured” in a sample when the presence or absence and/or quantity of said molecule or analyte or of said group of molecules or analytes is detected or determined in the sample, preferably substantially to the exclusion of other molecules and analytes.

The terms “quantity,” “amount,” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the molecule or analyte in a sample obtained from a healthy subject or, as appropriate, a sample obtained from a subject known to have IM or GC, including a particular type of GC. These values or ranges can be obtained from a single patient or from a group of patients.

A target analyte is differentially present between the two samples if the amount of the target analyte in one sample is statistically significantly different from the amount of the target analyte in the other sample. As used herein, the phrase “differentially expressed” refers to differences in the quantity and/or the frequency of a target analyte present in a sample taken from patients having, for example, a particular disease as compared to a control subject. For example, without limitation, a target analyte can be an antibody that is present at an elevated level or at a decreased level in samples of patients having a particular condition as compared to samples of control subjects. A target analyte can be differentially present in terms of quantity, frequency or both. In some cases, a target analyte is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively (or additionally), a target analyte is differentially present between the two sets of samples if the frequency of detecting the target analyte in samples of patients suffering from a particular disease or condition is statistically significantly higher or lower than in the control samples. For example, a target analyte is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

An antibody molecule that can “bind”, “bind to”, “specifically bind”, or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein.

Generally, the term “specificity” refers to the number of different types of antigens or epitopes to which a particular antigen-binding molecule or antigen-binding protein (such as an immunoglobulin, an antibody, an immunoglobulin single variable domain) can bind. The specificity of an antigen-binding protein can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding protein: the lesser the value of the KD, the stronger the binding strength between an epitope and the antigen-binding molecule (alternatively, the affinity can also be expressed as the affinity constant (KA), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as an antibody of the invention) and the pertinent antigen. Avidity is related to both the affinity between an epitope and its antigen binding site on the antigen-binding molecule and the number of pertinent binding sites present on the antigen-binding molecule.

As used in this specification and the claims, the singular forms “a,” “an,” and “the” include plural forms unless the context clearly dictates otherwise. Thus, the indefinite articles “a” and “an,” as used herein in the specification and in the claims should be understood to mean “at least one”, unless clearly indicated to the contrary.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term. Where ranges are stated, the endpoints are included within the range unless otherwise stated or otherwise evident from the context.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.” Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention. No language in the specification should be construed as indicating any non-claimed element as essential to the practice of the invention. It should be understood that descriptions of exemplary embodiments are not intended to limit the invention to the particular forms disclosed, but on the contrary, the intention is to cover all modifications, equivalents and alternatives falling within the spirit and scope of the invention as defined by the appended claims. It should also be understood that, unless clearly indicated to the contrary, in any methods claimed herein that include more than one step or act, the order of the steps or acts of the method is not necessarily limited to the order in which the steps or acts of the method are recited. All methods described herein can be performed in any suitable order unless otherwise indicated herein or otherwise clearly contradicted by context.

### EXAMPLES

The following Examples are illustrative and are not intended to limit the scope of the claimed subject matter.

### Example 1

### Identification of Anti-Helicobacter Pylori Antibody Signatures in Gastric Intestinal Metaplasia (IM)

Chronic Helicobacter pylori infection may induce gastric intestinal metaplasia (IM), which increases gastric cancer risk. The combination of H. pylori-specific antibodies with other serologic biomarkers may identify individuals with intestinal metaplasia to triage for endoscopic screening and surveillance. A protein array was used to scan about 90% of the H. pylori proteome and identify antibodies associated with gastric intestinal metaplasia. Anti-H. pylori antibody profiles between IM cases and non-atrophic gastritis (NAG) controls were compared. Humoral responses to 1528 H. pylori proteins were evaluated among a discovery set of 50 IM and 50 NAG using H. pylori protein arrays. Antibodies with ≥20% sensitivity at 90% specificity for either group were selected and further validated blindly in an independent set of 100 IM and 100 NAG using odds ratios (OR). A validated multi-signature was evaluated using the area under the receiver operating characteristics curve (AUC) and net reclassification improvement (NRI). A two-stage study in a high gastric cancer risk population found moderate-to-strong novel associations between anti-Helicobacter pylori antibodies and gastric intestinal metaplasia. These findings may have etiologic and clinical implications.

Here, the inventors developed and validated an H. pylori proteome array to assess anti-H. pylori humoral response profiles that scan ˜90% of the complete bacterial proteome.⁵ In this study, H. pylori protein arrays were used on samples from IM patients and NAG controls to evaluate the potential utility of specific anti-H. pylori antibodies as a non-endoscopic diagnosis and to advance the understanding of the etiological role of H. pylori in the progression to IM. To further explain the dynamics of H. pylori antibodies, the inventors also compared immunoproteomic profiles of current and past H. pylori infections.

Materials and Methods:

Study Population

Adults included in this study are part of an endoscopic campaign conducted between April and May 2017 in a high gastric cancer risk area of Chile. All individuals with gastrointestinal symptoms were referred for an upper endoscopy at the Intercultural Hospital of Nueva Imperial. From all available patients with histologically confirmed IM and NAG, the inventors randomly selected discovery (50 IM and 50 NAG) and validation (100 IM and 100 NAG) sets (Table 2). A global IM diagnosis was based on five biopsies collected using the Sydney protocol. Approximately 50% of the IM cases in both sample sets (52% discovery and 47% validation) represent advanced stages (i.e., extension to corpus). IM cases and NAG controls were comparable in H. pylori seropositivity as determined by whole-cell enzyme-linked immunosorbent assay (wcELISA; Biohit), H. pylori positivity by histology (Giemsa stain; using the five biopsies), and H. pylori positivity by urease (Pronto Dry®; using an antral biopsy with a final reading at 60 min.). CagA seropositivity (CagA-ELISA; Genesis Diagnostics) was higher in IM cases as compared to NAG controls. A past H. pylori infection was defined as a positive result for either wcELISA or CagA-ELISA with a negative result for both histology and urease. A current H. pylori infection was defined as a positive result for either histology or urease, regardless of results on wcELISA and CagA-ELISA. Overall positivity was defined as positivity for either past or current infection.

The original endoscopy campaign was approved by the Institutional Review Boards of the Chilean Pontificia Universidad Catolica and the U.S. National Cancer Institute. Informed consent was obtained from all participants. The present study was exempted by the U.S. National Institutes of Health Office of Human Subjects Research Protection from institutional review board evaluation.

TABLE 2
Selected characteristics of gastric intestinal metaplasia
(IM) patients and non-atrophic gastritis (NAG) controls.

| | Discovery set | | Validation set | |
|---|---|---|---|---|
| | IM | NAG | IM | NAG |
| | cases | controls | cases | controls |
| | (n = 50) | (n = 50) | (n = 100) | (n = 100) |
| Age in years, median (range) | 60 | 55 | 59 | 54 |
| | (35-73) | (40-76) | (39-79) | (39-85) |
| Male sex, n (%) | 26 (52) | 14 (28) | 36 (36) | 23 (23) |
| H. pylori seropositivity¹, | 27 (54) | 28 (56) | 61 (61) | 62 (62) |
| n (%) | | | | |
| CagA serology, n (%) | 37 (74) | 19 (38) | 74 (74) | 41 (41) |
| H. pylori histology², n (%) | 19 (38) | 21 (42) | 47 (47) | 49 (49) |
| H. pylori urease test, n (%) | 21 (42) | 20 (40) | 49 (49) | 48 (48) |
| Overall H. pylori infection³, | 43 (86) | 37 (74) | 90 (90) | 86 (86) |
| n (%) | | | | |
| past H. pylori infection⁴, | 19 (38) | 15 (30) | 34 (34) | 32 (32) |
| n (%) | | | | |
| current H. pylori infection⁵, | 24 (48) | 22 (44) | 56 (56) | 54 (54) |
| n (%) | | | | |

¹ELISA tested with H. pylori whole-cell lysate (wcELISA)
²Giemsa histology stain for H. pylori
³Positivity for either past or current H. pylori infection.
⁴wcELISA(+) or CagA-ELISA(+) with histology(−) and urease(−) among individuals with overall H. pylori infection
⁵Histology(+) or urease(+), regardless of results on wcELISA and CagA-ELISA among individuals with overall H. pylori infection

Selection of H. Pylori Genes

A total of 1528 full-length H. pylori genes were obtained in Gateway Entry clones from the U.S. Biodefense and Emerging Infections Research Resources Repository, including 1454 genes from the strain 26695 (covering 91% of the full proteome) and 74 genes from the strain J99 (12 genes with homology >90% between these two reference strains). CagA was PCR cloned from full-length (P12 strain) and a fragment comprising residues 1 to 884 (26695 strain).⁶ All genes were transferred into the pANT7-cGST expression vector using recombinational cloning. VacA gene was not available in the inventors' clone library and hence anti-VacA was not assessed in this study.

Fabrication, Expression, and Probing of H. Pylori-NAPPA Arrays

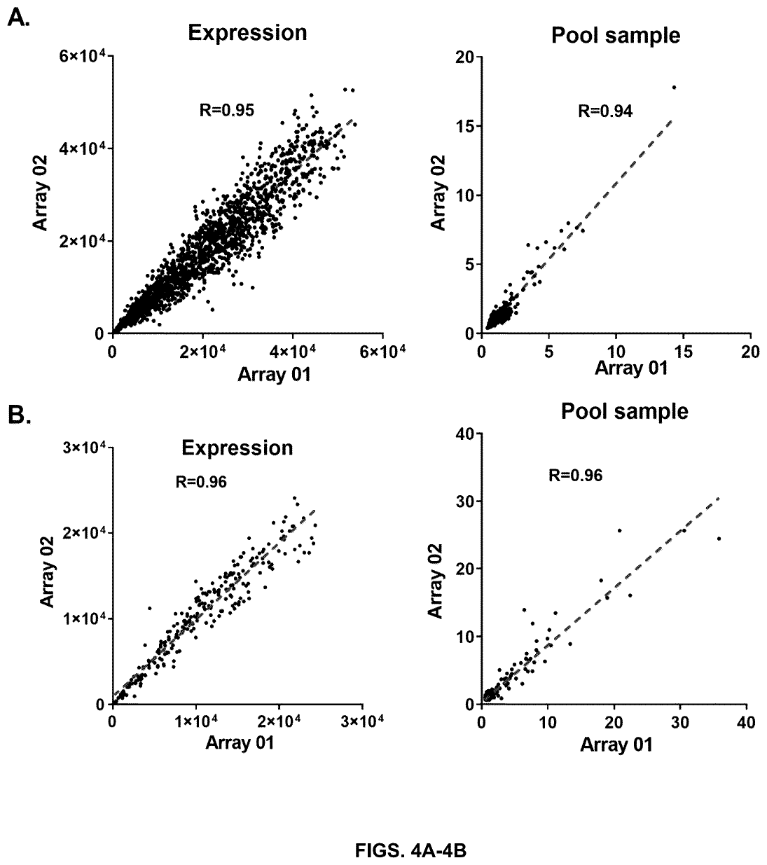

The inventors developed a 1528-protein H. pylori-NAPPA array to scan all bacterial antigens. Based on the results of the 1528-protein array in the discovery sample set, a smaller 245-protein H. pylori-NAPPA was created that includes 62 immunogenic antigens for which antibodies were present in >10% of IM cases or NAG controls and 183 proteins for which antibodies were absent in either group (i.e., 0% seroprevalence) to be used for the array data normalization. Both arrays were fabricated as previously reported.^{5, 7} Briefly, plasmid DNA for H. pylori expression clones into silicon nano-well substrates using a piezoelectric dispensing system. At the time of usage, NAPPA arrays were incubated with cell-free protein expression lysates at 30° C. for 2 hours and 15° C. for 30 minutes for in vivo protein expression and in situ capture. Isotype-specific (Immunoglobulin, IgG and IgA) antibody profiling was performed by incubating the arrays with 1:100 dilution of serum followed by detection with Alexa 647 labeled goat anti-human IgG and Cy3 labeled goat anti-human IgA. NAPPA arrays were scanned on a Tecan PowerScanner and raw fluorescence intensities were extracted using ArrayPro Analyzer Software. The reproducibility of the protein display on NAPPA arrays was assessed using an anti-GST antibody on duplicates, for which inter-array correlation coefficients were 0.95 for discovery and 0.96 for validation arrays. More than 99.7% (discovery sample set) and 100% (validation set) of H. pylori proteins were well-expressed with raw fluorescence intensities higher than the median intensity of all negative control spots plus 3 times standard deviations. A pooled serum combining all samples in the discovery set was used as an internal positive control to assess the antibody profiling reproducibility and resulted in inter-array correlations of 0.94 for discovery and 0.96 for validation arrays, respectively (FIG. 4B). Blinded testing was performed on all study samples. Antibody responses on both H. pylori-NAPPA arrays were normalized as Median Normalized Intensity (MNI) via dividing the raw values by the median signal intensity of all proteins within each array. Seropositivity was defined as MNI >2.0.

Anti-H. Pylori Antibodies in the Discovery and Validation Sample Sets

The discovery sample set (50 IM and 50 NAG) was profiled using both 1528-protein and 245-protein H. pylori-NAPPAs. There were high correlations (median R-value, 0.89) of the MNI values between the 1528- and 245-protein H. pylori-NAPPAs for the overlapping 245 proteins tested in the discovery set. The validation sample set (100 IM and 100 NAG) was probed only on the 245-protein H. pylori-NAPPA. Relative seroprevalence was calculated as the percentage of IM cases or NAG controls with MNI exceeding the 90 th percentile of the other group. Antibodies with relative seroprevalence ≥20% in either IM cases or controls (in the discovery set) were selected as candidate antibodies for validation of their performance in distinguishing IM cases from NAG controls in the validation set.

Statistical Analyses

The differences in the seroprevalence in IM cases and NAG controls, or between positive and negative results from wcELISA, CagA-ELISA, histology, and urease tests by case-control status were assessed by the Wilcoxon rank-sum test. Kappa coefficients measured agreement between available wcELISA and overall positivity results for the top-five most immunoreactive antibodies, as well as CagA-ELISA and CagA-NAPPA. For the discovery set, the sensitivity and specificity were computed for each antibody comparing IM and NAG, and antibodies with ≥20% sensitivity at 90% specificity for either direction were selected for further evaluation using the validation set. When assessing the performance of each selected antibody in the validation set, sensitivity and specificity were computed using the cutoff value from the discovery set and unadjusted odds ratio (OR) and p-value were calculated by the Chi-square tests. Antibodies with OR<0.5 or >2.0 with p-value <0.05 were considered as validated antibodies.

The area under the receiver operating characteristic (ROC) curve (AUC) values were calculated for each validated antibody using logistic regression. Correlations among the validated antibodies were assessed by Pearson correlation tests. Net reclassification improvement (NRI) was used to evaluate reclassification of the validated model, compared to an anti-CagA only model. In addition, a case-case comparison of corpus extension and antral restricted IM was run in both discovery and validation sample sets. The comparison between past vs. current infection among IM cases and NAG controls combined was conducted following a similar analytical approach. All tests were two-sided and p-values <0.05 were considered statistically significant. Analyses were conducted with R version 3.6 (R Core Team, R Foundation for Statistical Computing, Vienna, Austria), Stata version 15 (Stata Corp, College Station, TX, USA), and GraphPad Prism 8.0.2 (GraphPad Software, Inc., CA, USA).

Results:

Anti-H. Pylori Antibody Response and Prevalence in IM Cases and NAG Controls in the Discovery Sample Set

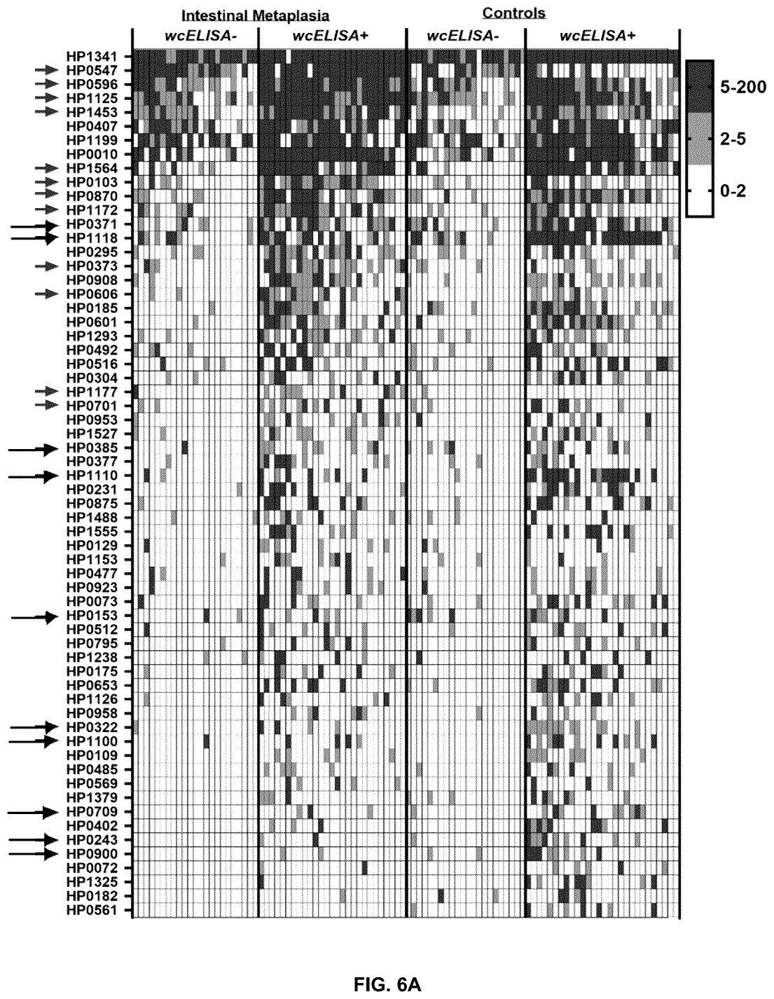

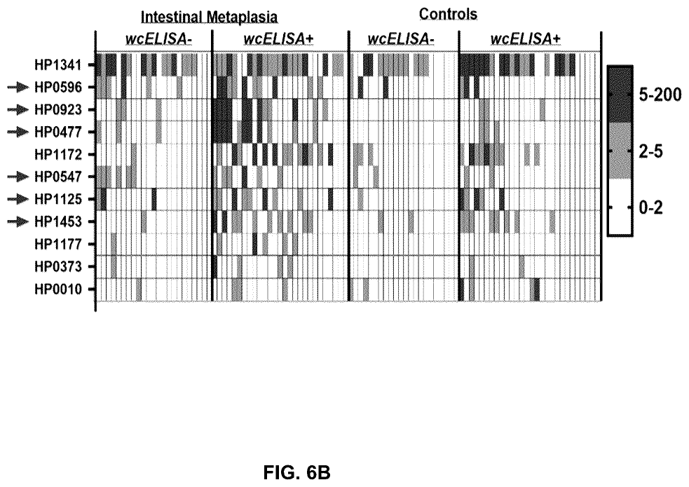

Of all antibodies profiled on the 1528-protein H. pylori-NAPPA, 62 IgG and 11 IgA antibodies showed >10% seropositivity in IM and/or NAG groups of the discovery sample set (Table 3, FIG. 6). The proteins targeted by the 11 IgA antibodies also elicited strong reactive IgG antibodies. In IM cases and NAG controls combined, anti-HP1341/TonB/TonB2 (seropositivity=98%), anti-HP1125/PalA/Omp18 (83%), anti-HP0596/Tipα (78%), anti-HP1199/RpI2/Rp17/RpIL (72%), anti-HP0010/GroEL/HSPb/Hsp60/MopA (70%), and anti-HP0547/CagA (70%) showed the highest overall seropositivity. Combining the results of wcELISA, cagA-ELISA, histology and urease, the overall positivity of H. pylori was 80% in the discovery sample set.

TABLE 3
Seropositivity of 62 IgG and 11 IgA antibodies on 1528-gene H. pylori NAPPA in intestinal metaplasia cases (IM)
and non-atrophic gastritis controls (NAG) in the discovery and validation sets.

| | | | Discovery | | | | Validation | | | |
|---|---|---|---|---|---|---|---|---|---|---|
| | | | IgG, % | | IgA, % | | IgG, % | | IgA, % | |
| | | | IM | NAG | IM | NAG | IM | NAG | IM | NAG |
| Protein | Common name | Full Name | (n = 50) | (n = 50) | (n = 50) | (n = 50) | (n = 100) | (n = 100) | (n = 100) | (n = 100) |
| HP1341 | TonB2, TonB | Energy transducer | 98 | 98 | 70 | 62 | 97 | 100 | 57 | 64 |
| HP0547 | CagA | Cytotoxicity-associated | 90 | 50 | 24 | 8 | 88 | 47 | 14 | 23 |
| | | immunodominant antigen | | | | | | | | |
| HP0596 | Tipα | Tumor necrosis factor | 84 | 72 | 32 | 12 | 90 | 74 | 27 | 12 |
| | | alpha-inducing protein | | | | | | | | |
| HP1125 | PalA/Omp18 | Peptidoglycan associated lipoprotein | 84 | 82 | 24 | 12 | 90 | 82 | 30 | 15 |
| HP1453 | HomD | Membrane protein | 76 | 56 | 22 | 20 | 75 | 64 | 19 | 22 |
| HP1199 | RpI2, Rpl7, RpIL | 50S ribosomal protein L7/L12 | 70 | 74 | 4 | 8 | 66 | 69 | 8 | 6 |
| HP0407 | BisC | Biotin sulfoxide reductase | 72 | 62 | 4 | 4 | 65 | 68 | 5 | 9 |
| HP0010 | GroEL, HSPb, | Molecular chaperone | 70 | 70 | 8 | 12 | 75 | 84 | 11 | 12 |
| | Hsp60, MopA | | | | | | | | | |
| HP1118 | Ggt | Gamma-glutamyltranspeptidase | 44 | 64 | 0 | 6 | 50 | 62 | 5 | 10 |
| HP0371 | AccB, FabE | Biotin carboxyl carrier protein | 48 | 56 | 0 | 4 | 48 | 70 | 1 | 8 |
| HP1564 | PlpA | ABC transporter substrate-binding | 56 | 56 | 0 | 0 | 67 | 58 | 1 | 0 |
| | | protein | | | | | | | | |
| HP0870 | FlgE | Flagellar hook protein | 50 | 56 | 0 | 2 | 57 | 65 | 2 | 5 |
| HP0103 | TlpB | Methyl-accepting chemotaxis | 52 | 26 | 4 | 4 | 49 | 23 | 3 | 3 |
| | | protein | | | | | | | | |
| HP1172 | GlnH | GlutamineABC transporter | 50 | 32 | 28 | 26 | 44 | 43 | 30 | 25 |
| | | substrate-binding protein | | | | | | | | |
| HP0295 | Fla | Flagellin B homolog | 44 | 30 | 2 | 0 | 44 | 36 | 1 | 0 |
| HP0373 | | Uncharacterized protein | 42 | 20 | 10 | 4 | 26 | 21 | 3 | 1 |
| HP0185 | | Hypothetical protein | 32 | 42 | 6 | 8 | 30 | 37 | 4 | 12 |
| HP0601 | FlaA | Flagellin A | 32 | 38 | 4 | 8 | 41 | 45 | 7 | 11 |
| HP1110 | PorA | Pyruvate flavodoxin oxidoreductase | 18 | 36 | 0 | 0 | 27 | 27 | 0 | 0 |
| | | subunit alpha | | | | | | | | |
| HP0908 | FlgE | Flagellar hook protein | 36 | 34 | 0 | 0 | 32 | 30 | 0 | 1 |
| HP0606 | MtrC | Membrane fusion protein | 34 | 16 | 0 | 0 | 28 | 29 | 0 | 1 |
| HP0516 | HslU | ATP-dependent protease | 22 | 32 | 2 | 0 | 13 | 26 | 0 | 0 |
| | | ATP-binding subunit | | | | | | | | |
| HP1293 | RpoA | DNA-directed RNA polymerase | 30 | 26 | 4 | 0 | 28 | 23 | 2 | 1 |
| | | subunit alpha | | | | | | | | |
| HP0073 | UreA | Urease subunit alpha | 14 | 28 | 4 | 2 | 17 | 22 | 0 | 2 |
| HP0492 | | Neuraminyllactose-binding | 28 | 22 | 0 | 2 | 23 | 20 | 4 | 2 |
| | | hemagglutinin | | | | | | | | |
| HP0385 | | Hypothetical protein | 20 | 26 | 6 | 6 | 24 | 43 | 5 | 9 |
| HP0653 | FtnA, Pfr | Bacterial non-heme ferritin | 12 | 26 | 0 | 0 | 17 | 23 | 1 | 2 |
| HP0322 | ChePep | Chemotaxis regulatory protein | 10 | 24 | 0 | 0 | 19 | 11 | 1 | 2 |
| HP0153 | RecA | Recombinase | 14 | 24 | 0 | 0 | 20 | 32 | 0 | 6 |
| HP0304 | | Alginate_lyase domain-containing | 22 | 24 | 0 | 2 | 16 | 25 | 0 | 0 |
| | | protein | | | | | | | | |
| HP0231 | | Protein-disulfide isomerase | 18 | 24 | 4 | 2 | 15 | 29 | 0 | 0 |
| HP0875 | KatA | Catalase | 18 | 24 | 0 | 0 | 21 | 35 | 1 | 0 |
| HP1177 | Omp27 | Outer membrane protein | 22 | 2 | 12 | 0 | 20 | 3 | 10 | 1 |
| HP1100 | Edd | Phosphogluconate dehydratase | 10 | 22 | 0 | 2 | 5 | 11 | 0 | 1 |
| HP0701 | GyrA | DNA gyrase subunit A | 22 | 18 | 0 | 0 | 18 | 16 | 0 | 1 |
| HP0953 | | Uncharacterized protein | 22 | 14 | 0 | 0 | 16 | 8 | 0 | 0 |
| HP1527 | ComH | Hypothetical protein | 22 | 18 | 0 | 4 | 25 | 20 | 4 | 4 |
| HP0243 | NapA | DNA protection during starvation | 4 | 20 | 0 | 0 | 11 | 24 | 0 | 0 |
| | | protein | | | | | | | | |
| HP0709 | | Uncharacterized protein | 6 | 20 | 0 | 0 | 9 | 25 | 0 | 0 |
| HP0900 | HypB | Hydrogenase/urease maturation | 4 | 20 | 0 | 0 | 5 | 13 | 1 | 1 |
| | | factor | | | | | | | | |
| HP0377 | DsbC | Thiol:disulfide interchange protein | 20 | 14 | 2 | 0 | 23 | 19 | 2 | 0 |
| HP1555 | Tfs | Elongation factor Ts | 16 | 20 | 0 | 0 | 18 | 20 | 1 | 1 |
| HP0402 | PheT | Phenylalanine-tRNA ligase beta | 6 | 18 | 0 | 2 | 10 | 13 | 2 | 1 |
| | | subunit | | | | | | | | |
| HP1488 | | Membrane protein | 18 | 10 | 0 | 4 | 15 | 15 | 1 | 0 |
| HP1379 | Ion | ATP-dependent protease | 8 | 18 | 0 | 0 | 20 | 14 | 2 | 3 |
| HP0512 | GlnA | Glutamine synthetase | 14 | 18 | 0 | 4 | 13 | 25 | 1 | 3 |
| HP1325 | FumC | Fumarate hydratase class II | 2 | 16 | 0 | 0 | 3 | 19 | 0 | 0 |
| HP0129 | | Hypothetical protein | 16 | 10 | 0 | 0 | 6 | 12 | 0 | 0 |
| HP0561 | FabG | 3-ketoacyl-ACP reductase | 0 | 16 | 0 | 0 | 6 | 5 | 0 | 0 |
| HP1153 | ValS, ValRS | Valine-tRNA ligase | 16 | 10 | 0 | 0 | 2 | 3 | 0 | 0 |
| HP0477 | HopJ, Omp12 | Membrane protein | 16 | 14 | 28 | 6 | 22 | 10 | 20 | 14 |
| HP0109 | DnaK, HSP70 | Chaperone protein | 10 | 16 | 0 | 0 | 11 | 13 | 0 | 0 |
| HP1126 | TolB | Tol-Pal system protein | 12 | 16 | 2 | 0 | 12 | 4 | 0 | 0 |
| HP0923 | HopK, Omp22 | Membrane protein | 16 | 14 | 30 | 6 | 22 | 11 | 21 | 12 |
| HP0795 | Tig | Trigger factor | 14 | 12 | 0 | 0 | 11 | 5 | 0 | 0 |
| HP0485 | | Catalase-related peroxidase | 10 | 14 | 2 | 0 | 3 | 7 | 0 | 1 |
| HP1238 | AmiF | Formamidase | 14 | 10 | 0 | 0 | 19 | 18 | 0 | 0 |
| HP0175 | PpiC | Peptidylproly1 isomerase | 14 | 14 | 0 | 0 | 8 | 18 | 0 | 1 |
| HP0958 | | Zf-RING_7 domain-containing | 12 | 10 | 2 | 0 | 7 | 9 | 2 | 1 |
| | | protein | | | | | | | | |
| HP0182 | LysS, LysRS | Lysine-tRNA ligase | 2 | 12 | 0 | 0 | 2 | 2 | 0 | 1 |
| HP0569 | YchF | Ribosome-binding ATPase | 10 | 6 | 0 | 0 | 5 | 6 | 0 | 0 |
| HP0072 | UreB | Urease subunit beta | 4 | 10 | 2 | 2 | 6 | 8 | 0 | 0 |

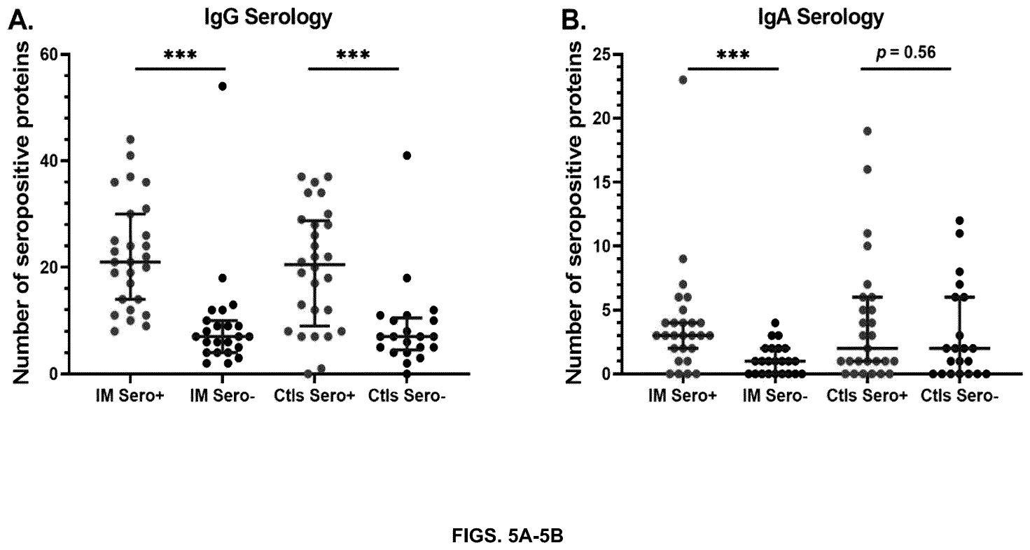

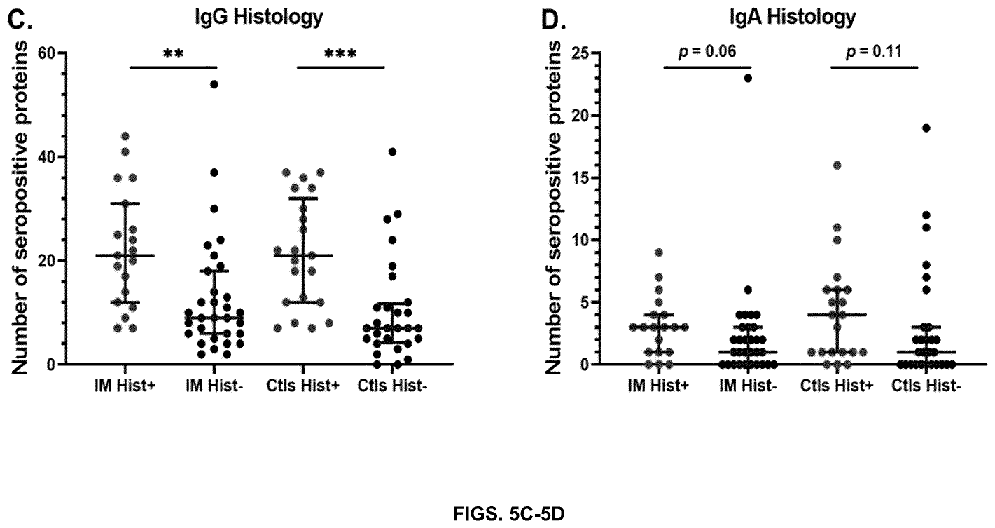

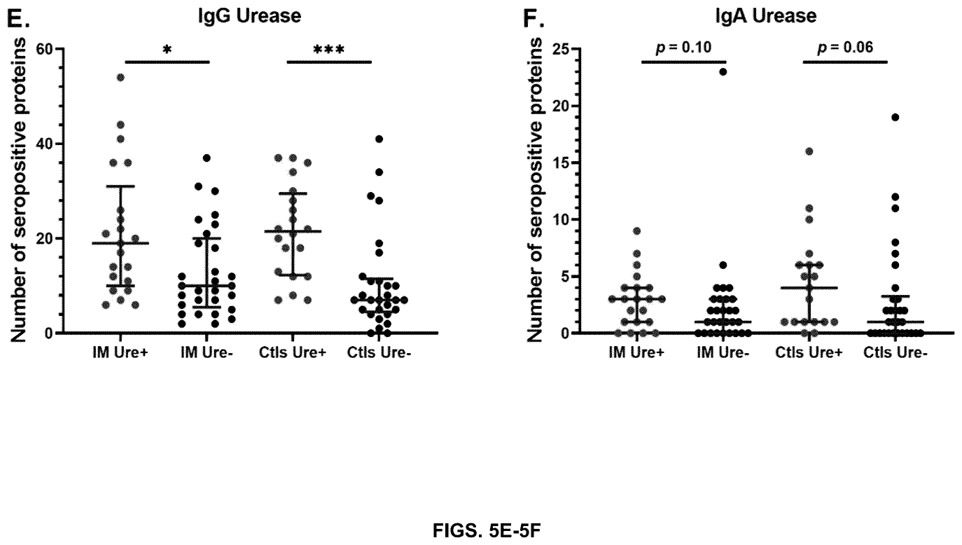

Regardless of the case-control status, H. pylori-positive individuals as defined by any of the 4 conventional tests (i.e., wcELISA, cagA-ELISA, histology, or urease) had a higher total number of IgG anti-H. pylori antibodies relative to H. pylori-negative individuals, while the number of IgA antibodies was comparable between the two groups (FIG. 5).

Among the top-five most reactive antibodies, seropositivity to anti-HP0010/GroEL exhibited the highest degree of agreement with wcELISA (Kappa coefficient=0.4), while there was low to moderate agreement between overall positivity and the other 4 antibodies (Kappa coefficient ranged from 0.05 to 0.3; Table 4). The degree of agreement between cagA-ELISA and seropositivity to HP0547/CagA on H. pylori-NAPPA was moderate (coefficient=0.6).

TABLE 4
Kappa coefficients for the agreement of wcELISA and overall
H. pylori positivity with each of the top-5 antibodies.

| Proteins | wcELISA | Overall positivity |
|---|---|---|
| HP1341/TonB/TonB2 | 0.02 | 0.05 |
| HP1125/PalA/Omp18 | 0.2* | 0.3* |
| HP0596/Tipα | 0.3* | 0.3* |
| HP1199/RpI2/Rpl7/RpIL | 0.2* | 0.1 |
| HP0010/GroEL/HSPb/Hsp60/MopA | 0.4* | 0.2* |

*p < 0.05

Discovery and Validation of Discriminatory Anti-H. Pylori Antibodies Between IM Cases and NAG Controls

Among the 73 antibodies (62 IgG and 11 IgA) identified on the 1528-protein H. pylori-NAPPA and re-tested on the 254-protein NAPPA, 12 IgG and 6 IgA antibodies showed higher relative seroprevalence in IM cases, and 10 IgG antibodies showed higher relative seroprevalence in NAG controls in the validation set (Table 5, FIG. 6). Five IgG antibodies (anti-HP0547/CagA, anti-HP1125/PalA/Omp18, anti-HP0596/Tipα, anti-HP1177/HopQ/Omp27, and anti-HP0103/TlpB) and two IgA antibodies (HP0596/Tipα and anti-HP1125/PalA/Omp18) were validated with higher sensitivity (i.e., significantly higher responses in IM cases) (Table 5). Six IgG antibodies (anti-HP0709, anti-HP0900/HypB, anti-HP0371/FabE/AccB, anti-HP0243/NapA, anti-HP0153/RecA, and anti-HP0385) were validated with higher specificity (i.e., lower responses in IM) (Table 5). The five IgG antibodies with higher reactivity in IM cases (than in controls) had AUC values ranging from 0.65-0.77, while the five IgG antibodies' lower reactivity in IM had AUC values ranging from 0.50-0.65. The validated two IgA antibodies higher in IM had AUC values of 0.57 and 0.60.

TABLE 5
Candidate and validated anti-H. pylori antibodies differential between intestinal metaplasia (IM) cases and non-atrophic
gastritis (NAG) controls in the 245-gene NAPPA

| | | | | | Discovery sample set | | Validation sample set | | | | |
|---|---|---|---|---|---|---|---|---|---|---|---|
| | | | | | | NAG | | NAG | | | |
| | | | | | IM cases | controls | IM cases | controls | | | |
| Ig | | Common | | | (n = 50)¹, | (n = 50)², | (n = 100)¹, | (n = 100)², | | | |
| Isotype | Protein | names | Full name | Profile | % | % | % | % | OR | p-value | AUC |
| IgG | HP1177 | Omp27 | Outer membrane protein | High | 22 | 2 | 20 | 3 | 8.08 | <0.001 | 0.73 |
| | HP0547 | CagA | Cytotoxicity-associated | in IM | 48 | 10 | 45 | 15 | 4.64 | <0.001 | 0.77 |
| | | | immunodominant antigen | | | | | | | | |
| | HP0596 | Tipα | Tumor necrosis factor | | 30 | 10 | 23 | 7 | 3.97 | 0.002 | 0.66 |
| | | | alpha-inducing protein | | | | | | | | |
| | HP0103 | TlpB | Methyl-accepting chemotaxis | | 22 | 10 | 25 | 8 | 3.83 | 0.001 | 0.68 |
| | | | protein | | | | | | | | |
| | HP1125 | PalA/ | Peptidoglycan associated | | 32 | 10 | 37 | 16 | 3.08 | 0.001 | 0.65 |
| | | OMP18 | lipoprotein | | | | | | | | |
| | HP0908 | FlgE | Flagellar hook protein | | 20 | 10 | 17 | 8 | 2.36 | 0.054 | 0.57 |
| | HP0701 | GyrA | DNA gyrase subunit A | | 20 | 10 | 16 | 10 | 1.71 | 0.207 | 0.51 |
| | HP1172 | GlnH | Glutamine ABC transporter | | 26 | 10 | 26 | 18 | 1.60 | 0.172 | 0.52 |
| | | | substrate-binding protein | | | | | | | | |
| | HP1453 | HomD | Membrane protein | | 20 | 10 | 16 | 12 | 1.40 | 0.415 | 0.55 |
| | HP0373 | | Uncharacterized protein | | 38 | 10 | 22 | 17 | 1.38 | 0.372 | 0.53 |
| | HP1564 | PlpA | ABC transporter | | 26 | 10 | 13 | 10 | 1.34 | 0.506 | 0.56 |
| | | | substrate-binding protein | | | | | | | | |
| | HP0606 | MtrC | Membrane fusion protein | | 28 | 10 | 22 | 19 | 1.20 | 0.599 | 0.56 |
| | HP0709 | | Uncharacterized protein | Low | 6 | 20 | 9 | 25 | 0.30 | 0.003 | 0.61 |
| | HP0900 | HypB | Hydrogenase/urease | in IM | 4 | 20 | 5 | 13 | 0.35 | 0.048 | 0.50 |
| | | | maturation factor | | | | | | | | |
| | HP0371 | AccB, | Biotin carboxyl carrier protein | | 10 | 20 | 9 | 21 | 0.37 | 0.017 | 0.65 |
| | | FabE | | | | | | | | | |
| | HP0243 | NapA | DNA protection during | | 4 | 20 | 11 | 24 | 0.39 | 0.016 | 0.63 |
| | | | starvation protein | | | | | | | | |
| | HP0385 | | Hypothetical protein | | 10 | 20 | 18 | 35 | 0.41 | 0.006 | 0.57 |
| | HP0153 | RecA | Recombinase | | 10 | 22 | 17 | 30 | 0.48 | 0.030 | 0.55 |
| | HP1100 | Edd | Phosphogluconate dehydratase | | 10 | 22 | 5 | 11 | 0.43 | 0.118 | 0.55 |
| | HP1118 | Ggt | Gamma-glutamyltranspeptidase | | 10 | 34 | 25 | 33 | 0.68 | 0.213 | 0.58 |
| | HP1110 | PorA | Pyruvate flavodoxin | | 10 | 30 | 24 | 16 | 1.66 | 0.157 | 0.54 |
| | | | oxidoreductase subunit alpha | | | | | | | | |
| | HP0322 | ChePep | Chemotaxis regulatory protein | | 10 | 24 | 19 | 11 | 1.90 | 0.113 | 0.42 |
| IgA | HP0596 | Tipα | Tumor necrosis factor | High | 26 | 10 | 20 | 9 | 2.53 | 0.027 | 0.57 |
| | | | alpha-inducing protein | in IM | | | | | | | |
| | HP1125 | PalA/ | Peptidoglycan associated | | 20 | 10 | 17 | 7 | 2.72 | 0.030 | 0.60 |
| | | OMP18 | lipoprotein | | | | | | | | |
| | HP0923 | HopK, | Membrane protein | | 30 | 6 | 21 | 12 | 1.95 | 0.086 | 0.58 |
| | | Omp22 | | | | | | | | | |
| | HP0547 | CagA | Cytotoxicity-associated | | 24 | 8 | 14 | 23 | 0.54 | 0.101 | 0.52 |
| | | | immunodominant antigen | | | | | | | | |
| | HP0477 | HopJ, | Membrane protein | | 28 | 6 | 20 | 14 | 1.54 | 0.259 | 0.52 |
| | | Omp12 | | | | | | | | | |
| | HP1453 | HomD | Membrane protein | | 20 | 10 | 15 | 16 | 0.93 | 0.845 | 0.50 |

¹Cutoff generated from the 90^{th} percentile of NAG controls
²Cutoff generated from the 90^{th} percentile of IM cases
Abbreviation: AUC = area under the curve; OR = odds ratio
Bolded numbers are for proteins that were validated, i.e., signification in the validation set

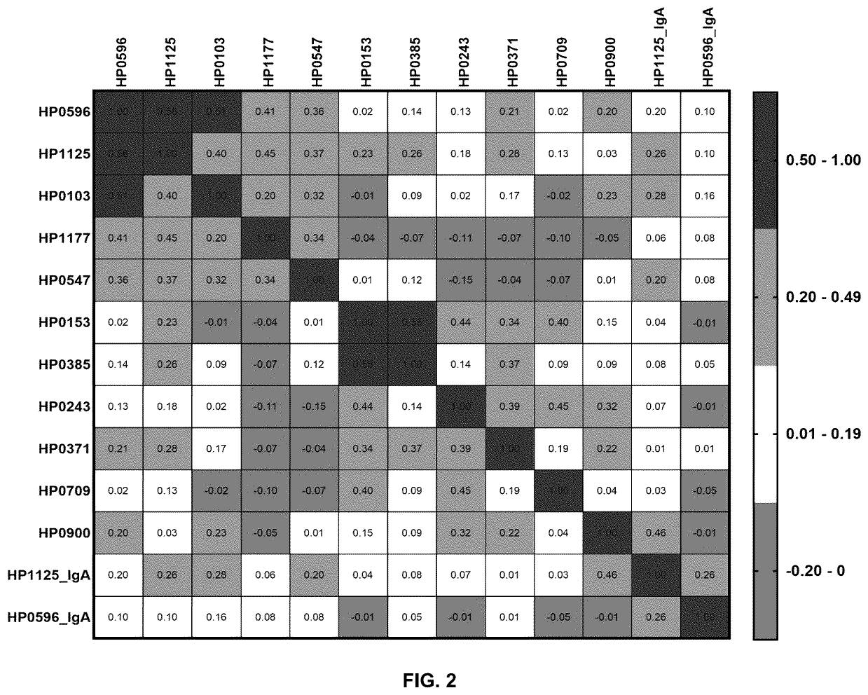

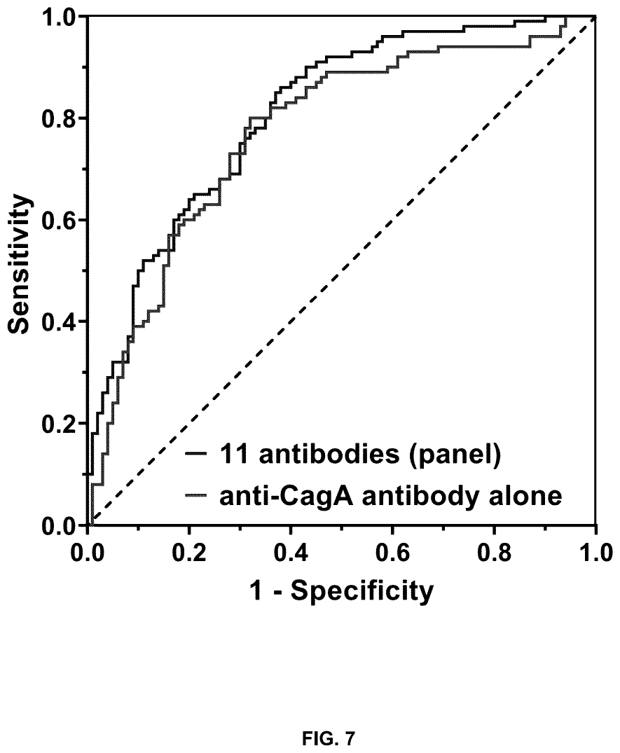

All pairwise correlations among the 13 IgG and IgA antibodies in the validation sample set are shown in FIG. 2. Among the five antibodies with higher seroprevalence in IM cases than in NAG controls, correlation coefficients ranged from 0.20 (anti-HP1177 vs. anti-HP0103) to 0.56 (anti-HP1125 vs. anti-HP0596). Amongst antibodies with lower seroprevalences in IM cases than in NAG controls, correlation coefficients ranged from 0.04 (anti-HP0709 vs. anti-HP0900) to 0.55 (anti-HP0153 vs. anti-HP0385). The correlations between IgG and IgA antibodies against the same antigens (anti-HP1125 and anti-HP0596) were weak (correlation coefficient <0.3). AUC for an antibody panel with all 13 validated IgG and IgA antibodies was 0.82 (95% CI, 0.76-0.87) distinguishing IM from NAG. A model including the 11 validated IgG antibodies (AUC =0.81; 95% CI, 0.75-0.87) had a better classification performance compared to an anti-CagA only (AUC=0.77; 95% CI, 0.70-0.84) model (NRI=0.44; p-value=0.001) (FIG. 7). The validated antibodies included 11 IgG (anti-HP1177/Omp27/HopQ [OR=8.1, p<0.001], anti-HP0547/CagA [4.6,p<0.001], anti-HP0596/Tipα [4.0,p=0.002], anti-HP0103/TlpB [3.8,p=0.001], anti-HP1125/PalA/Omp18 [3.1, p=0.001], anti-HP0153/RecA [0.48, p=0.03], anti-HP0385 [0.41, p=0.006], anti-HP0243/TlpB [0.39, p=0.016], anti-HP0371/FabE [0.37, p=0.017], anti-HP0900/HypB/AccB [0.35, p=0.048], and anti-HP0709 [0.30, p=0.003]), and 2 IgA (anti-HP1125/PalA/Ompl8 [2.7, p=0.03] and anti-HP0596/Tipα [2.5, p=0.027]).

Anti-H. Pylori Antibodies by Anatomical Extension of IM

H. pylori seropositivity by wcELISA was similar (59% vs. 58%) between the case-case analysis of corpus extension (n=73) and antral (n=77) restricted IM subsite. Out of total proteins profiled on the 254-protein H. pylori-NAPPA, two IgG antibodies (anti-HP0516/HslU and anti-HP0385) and one IgA antibody (anti-HP1453/HomD) showed significantly higher seropositivity in antral restricted as compared to corpus extension IM (p values <0.05) (Table 6).

TABLE 6
Anti-H. pylori antibodies that are differential between corpus extension and antral
restricted IM subsite in the 245-gene NAPPA, without consideration the wcELISA result.

| | | | | | Seropositivity | Seropositivity | | | |
|---|---|---|---|---|---|---|---|---|---|
| Ig | | Common | | | (corpus, | (antral, | | | p- |
| Isotype | Protein | names | Full Name | Profile | n = 73, %) | n = 77, %) | OR | 95% CI | value |
| IgG | HP0516 | HsIU | ATP- | High in | 10 | 22 | 2.67 | 1.35-5.30 | 0.03 |
| | | | dependent | antral | | | | | |
| | | | protease | | | | | | |
| | HP0385 | | ATP- | | 15 | 30 | 2.40 | 1.34-4.30 | 0.02 |
| | | | binding | | | | | | |
| | | | subunit | | | | | | |
| | | | hypothetical | | | | | | |
| | | | protein | | | | | | |
| IgA | HP1453 | HomD | membrane | High in | 14 | 26 | 2.21 | 1.20-4.06 | 0.05 |
| | | | protein | antral | | | | | |

Discovery and Validation of Discriminatory Anti-H. Pylori Antibodies Between Current vs. Past H. Pylori Infection

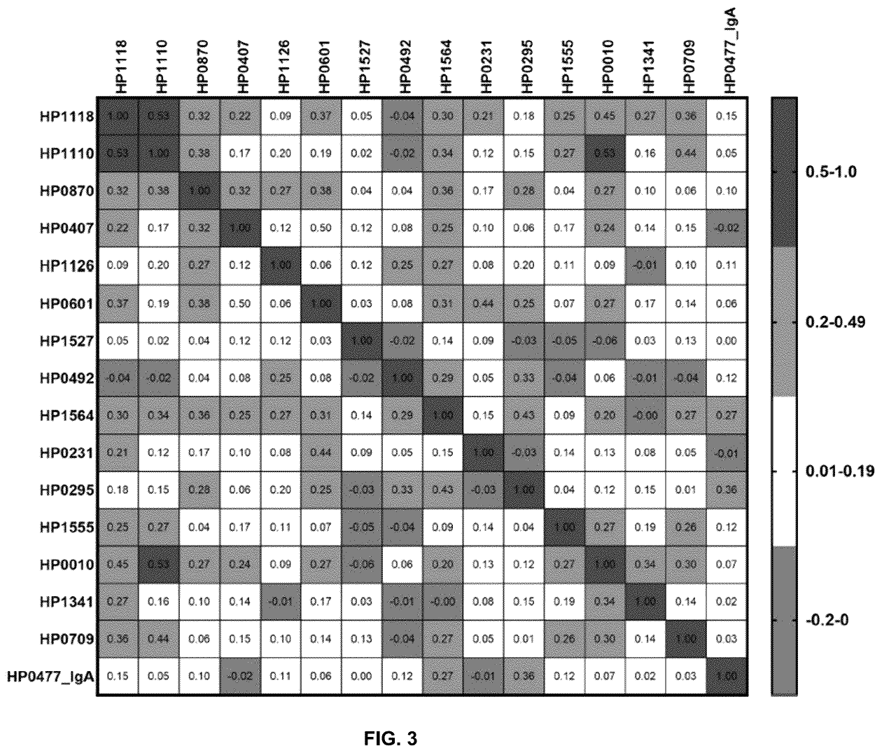

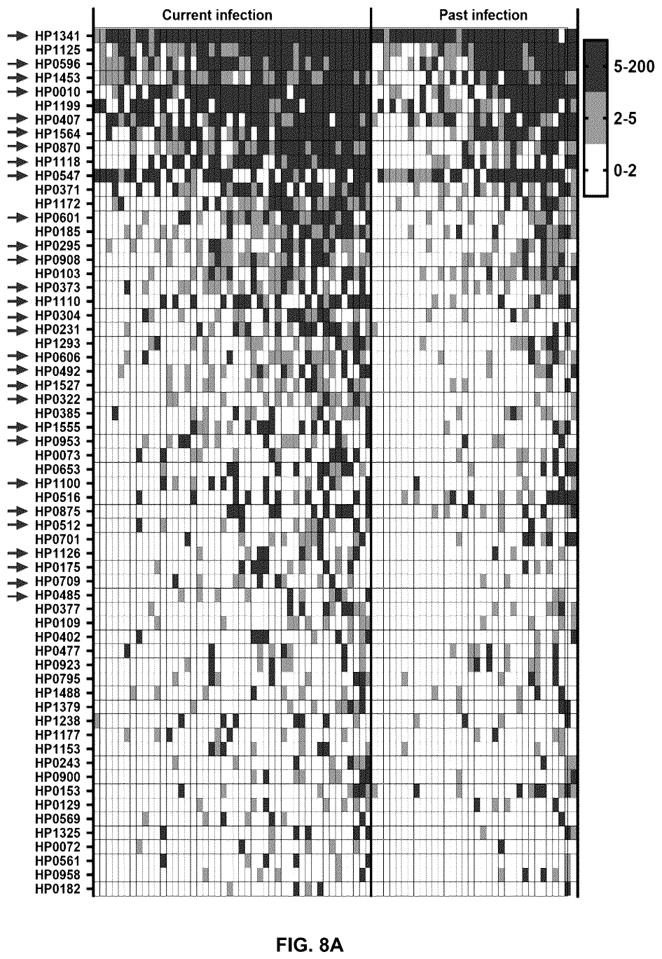

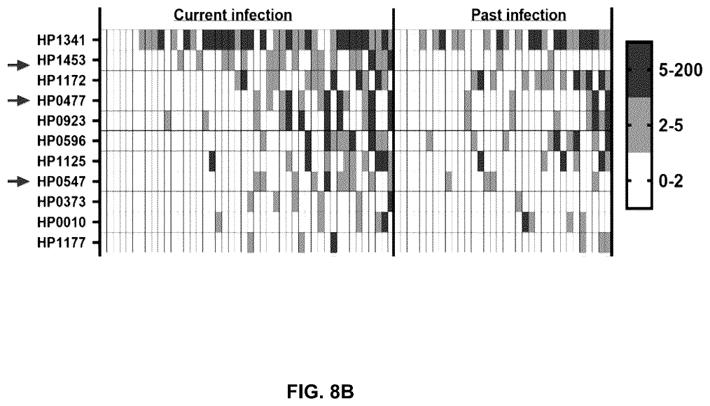

Prevalence of current and past H. pylori infections were similar in both discovery and validation sample sets (Table 2). Out of the 254 antibodies profiled, 15 IgG antibodies (anti-HP1118/Ggt, anti-HP1110/PorA, anti-HP0870/ FlgE, anti-HP0407/BisC, anti-HP0601/FlaA, anti-HP1126/TolB, anti-HP1527/ComH, anti-HP0492, anti-HP1564/PlpA, anti-HP0231, anti-HP0295/Fla, anti-HP1555/Tfs, anti-HP0010/GroEL, anti-HP0709, and anti-HP1341/TonB2, and one IgA (anti-HP0477/HopJ) were strongly significantly associated with current H. pylori infection with ORs ranging from 2.19 to 7.46, regardless of case-control status (Table 7, FIG. 8). All pairwise correlations among the 16 candidate IgG and IgA antibodies in the validation sample set are shown in FIG. 3. Correlation coefficients ranged from −0.06 (anti-HP0010 vs. anti-HP0492) to 0.53 (anti-HP1118 vs. anti-HP1110 and anti-HP0010 vs. anti-HP1110). AUC for a panel combining the validated 16 antibodies was 0.85 (95% CI, 0.79-0.90) distinguishing current from past H. pylori infection.

TABLE 7
Candidate and validated anti-H. pylori antibodies differential between current and past H. pylori infection in the 245-gene NAPPA.

| | | | | Discovery sample set¹ | | Validation sample set¹ | | | | |
|---|---|---|---|---|---|---|---|---|---|---|
| | | | | Current | Past | Current | Past | | | |
| | | | | infection | infection | infection | infection | | | |
| Ig | | Common | | (n = 46)², | (n = 34)², | (n = 110)², | (n = 60)², | | | |
| Isotype | Protein | names | Full name | % | % | % | % | OR | p-value | AUC |
| IgG | HP1118 | Ggt | Gamma-glutamyltranspeptidase | 39 | 12 | 43 | 9 | 7.46 | 0.000 | 0.72 |
| | HP1110 | PorA | Pyruvate flavodoxin oxidoreductase | 30 | 12 | 28 | 6 | 6.08 | 0.000 | 0.67 |
| | | | subunit alpha | | | | | | | |
| | HP0870 | FlgE | Flagellar hook protein | 37 | 12 | 35 | 9 | 5.49 | 0.000 | 0.70 |
| | HP0407 | BisC | Biotin sulfoxide reductase | 26 | 12 | 25 | 6 | 5.04 | 0.002 | 0.64 |
| | HP0601 | FlaA | Flagellin A | 48 | 12 | 51 | 18 | 4.67 | 0.000 | 0.71 |
| | HP1126 | TolB | Tol-Pal system protein | 22 | 9 | 13 | 3 | 4.67 | 0.030 | 0.69 |
| | HP1527 | ComH | Hypothetical protein | 26 | 12 | 31 | 9 | 4.47 | 0.001 | 0.71 |
| | HP0492 | | Neuraminyllactose-binding | 33 | 12 | 22 | 6 | 4.33 | 0.006 | 0.64 |
| | | | hemagglutinin | | | | | | | |
| | HP1564 | PlpA | ABC transporter substrate-binding | 22 | 12 | 16 | 5 | 4.11 | 0.019 | 0.76 |
| | | | protein | | | | | | | |
| | HP0231 | | Protein-disulfide isomerase | 39 | 9 | 32 | 11 | 3.93 | 0.001 | 0.69 |
| | HP0295 | Fla | Flagellin B homolog | 35 | 12 | 26 | 12 | 2.60 | 0.025 | 0.65 |
| | HP1555 | Tfs | Elongation factor Ts | 30 | 12 | 26 | 12 | 2.60 | 0.025 | 0.58 |
| | HP0010 | GroEL, HSPb, | Molecular chaperone | 33 | 12 | 23 | 11 | 2.48 | 0.044 | 0.61 |
| | | Hsp60, MopA | | | | | | | | |
| | HP0709 | | Uncharacterized protein | 22 | 6 | 23 | 11 | 2.48 | 0.044 | 0.58 |
| | HP1341 | TonB2, TonB | Energy transducer | 28 | 12 | 33 | 18 | 2.19 | 0.036 | 0.58 |
| | HP0304 | | Alginate_lyase domain-containing | 37 | 12 | 22 | 11 | 2.35 | 0.059 | 0.61 |
| | | | protein | | | | | | | |
| | HP0485 | | Catalase-related peroxidase | 22 | 6 | 8 | 2 | 5.79 | 0.064 | 0.60 |
| | HP0953 | | Uncharacterized protein | 30 | 12 | 17 | 8 | 2.55 | 0.070 | 0.62 |
| | HP0373 | | Uncharacterized protein | 33 | 12 | 21 | 11 | 2.23 | 0.078 | 0.61 |
| | HP1453 | HomD | Membrane protein | 22 | 12 | 16 | 8 | 2.39 | 0.094 | 0.60 |
| | HP0908 | FlgE | Flagellar hook protein | 24 | 12 | 17 | 9 | 2.09 | 0.132 | 0.68 |
| | HP0596 | Tipa | Tumor necrosis factor | 30 | 12 | 19 | 11 | 1.99 | 0.136 | 0.63 |
| | | | alpha-inducing protein | | | | | | | |
| | HP0512 | GlnA | Glutamine synthetase | 24 | 9 | 25 | 15 | 1.82 | 0.139 | 0.57 |
| | HP0371 | AccB, FabE | Biotin carboxyl carrier protein | 39 | 12 | 38 | 27 | 1.65 | 0.139 | 0.57 |
| | HP0547 | CagA | Cytotoxicity-associated | 22 | 12 | 20 | 12 | 1.81 | 0.178 | 0.50 |
| | | | immunodominant antigen | | | | | | | |
| | HP0322 | ChePep | Chemotaxis regulatory protein | 33 | 6 | 20 | 12 | 1.81 | 0.178 | 0.52 |
| | HP0175 | PpiC | Peptidylprolyl isomerase | 22 | 9 | 17 | 11 | 1.76 | 0.228 | 0.56 |
| | HP1100 | Edd | Phosphogluconate dehydratase | 24 | 12 | 9 | 5 | 2.10 | 0.264 | 0.51 |
| | HP0875 | KatA | Catalase | 22 | 12 | 25 | 18 | 1.46 | 0.325 | 0.53 |
| | HP0606 | MtrC | Membrane fusion protein | 22 | 12 | 16 | 15 | 1.10 | 0.831 | 0.56 |
| | HP0477 | HopJ, Omp12 | Membrane protein | 24 | 12 | 21 | 6 | 4.10 | 0.008 | 0.62 |
| IgA | HP1453 | HomD | Membrane protein | 39 | 6 | 27 | 17 | 1.88 | 0.107 | 0.68 |
| | HP0547 | CagA | Cytotoxicity-associated | 22 | 12 | 19 | 18 | 1.06 | 0.881 | 0.48 |
| | | | immunodominant antigen | | | | | | | |

¹Intestinal metaplasia cases and non-atrophic gastritis controls combined
²Cutoff generated from the 90^{th} percentile of past infection
Abbreviation: AUC = area under the curve; OR-odds ratio

Discussion:

This two-stage study represents the most comprehensive proteome-level analysis to identify anti-H. pylori antibodies to distinguish gastric IM from NAG. The inventors found moderate-to-strong associations with IM for 11 IgG and 2 IgA antibodies against several outer membrane proteins (OMPs) and proteins essential for bacterial survival. A combined model of the 11 IgG antibodies moderately discriminated IM from NAG. Another unique feature of the study was the comparison between current and past H. pylori infections. The inventors identified 15 IgG and 1 IgA antibodies associated with current infection, including responses to several proteins related to colonization and persistence.

H. pylori coevolved with humans and developed efficient mechanisms to avoid the

surveillance of the host immune system. As a consequence, H. pylori infection typically does not elicit a strong humoral response,⁸ which is consistent with the limited number of immunogenic proteins observed in this study (4%; 62/1528). Also, expanding the inventors' previous findings in gastric cancer using the same NAPPA array,⁵ the inventors confirmed that IgG antibodies were more informative in determining gastric pathology compared with IgA antibodies.

Anti-CagA antibodies are sustained for a long time even after successful H. pylori eradication.^{9, 10} A potential mechanism for the persistent antigenic stimuli may be due to the remnants of CagA-positive strains in the deepest gastric glands surviving long years of infection. The finding that a higher CagA seropositivity was moderately discriminative between IM cases and NAG controls aligns with two previous cross-sectional analyses^{11, 12} within the Linxian

Nutrition Intervention Trial cohort and the inventors' study in a Mexican population. Pan et al., compared antibodies against HP0547/CagA, HP0887/VacA, GroEL, UreA, HcpC, and HP1118/Ggt in patients with precancerous lesions and those with superficial gastritis, and reported a positive IM association with anti-HP0547/CagA and an inverse IM association with anti-HP1118/gGT.¹¹ Using a different platform, a 15-plex Luminex fluorescent bead-based immunoassay, Epplein et al., reported positive associations of IM with antibodies against HP1564/Omp, HP0547/CagA, HP0887/VacA, HcpC, HP0305, GroEL, NapA, HyuA, Cad, and HpaA.¹² These studies confirmed the essential role of the oncoprotein CagA in gastric carcinogenesis.

H. pylori OMPs serve as key factors for nutrient scavenging and evasion of host defense mechanisms.¹³ In this study, two antibodies to OMPs were positively associated with IM. HP1177/Omp27/HopQ binds to carcinoembryonic antigen-related cell-adhesion molecules with high specificity.¹⁴ Type I HopQ may be associated with carcinogenesis by transferring CagA or triggering type IV secretion system to initiate and maintain chronic inflammation mainly by activating toll-like receptor 9 via canonical NF-κB pathway.^{15, 16} However, Taxauer et al., showed that the HopQ-CEACAM interaction is also important for activation of the non-canonical NF-κB pathway.¹⁷ HP1125/PalA/Omp 18 is expressed by all known H. pylori strains and is involved in persistent colonization.¹⁸ In particular, Omp 18 may alter interferon-y levels and optimize virulence phenotype (i.e, CagA), survive oxidative stress, and anti-phagocytosis. pH taxis is another important mechanism for the long-term persistence of H. pylori in the stomach.¹⁹ HP0103/TlpB is required in chemorepulsive responses to acid, as well as the quorum-sensing molecule autoinducer-2.²⁰ Interestingly, binding of polymeric G-repeats regulator to the upstream of tlpB is sufficient to regulate TlpB both at the transcript and protein leve.²¹ HP0596/Tipα is another tumor promoter secreted as dimers and enters the gastric cells, a process mediated through NF-κB activation,²² that also has DNA-binding activity.²³ Similar to CagA, antibodies to HP0596/Tipα may persist after bacterial eradication.²⁴ Interestingly, the analyses herein showed anti-HP0596/Tipα to be moderately positively correlated with other antibodies [HP1125/PalA/Omp18 and HP0103/TlpB). Although the best discriminatory model suggests independent effects for anti-HP1177/Omp27/HopQ, HP0547/CagA, HP0103/TlpB and HP1125/PalA/Omp18, given the complex pathway networks of antigens, their orchestrated synergistic contribution cannot be dismissed.

This study shows several inverse antibody associations with IM. These findings may indicate loss of antigenic stimuli potentially related to atrophy progression and/or use of antibiotics, or immune protection against carcinogenesis. In a previous NAPPA gastric cancer study, the inventors found several inverse associations including antibodies against HP0371/FabE/AccB, HP0243/NapA, and HP0153/RecA (marginal statistical significance) that were also identified in this IM study. HP0371/FabE/AccB is a biotin carboxyl carrier protein of acetyl-CoA carboxylase, which is involved in fatty acid biosynthesis.²⁵ NapA plays dual roles, recruiting host neutrophils/monocytes and stimulating the production of reactive oxygen intermediates, but on the other hand, sequestering iron and stress-resistant to oxidative stress.²⁶ NapA may also promote the formation of H₂O₂-induced biofilm and contribute to multidrug resistance.²⁷ RecA is a protein that is necessary for repairing DNA damage or facilitating recombination. In addition, mutants of H. pylori recA are hypersensitive to DNA-damaging agents such as metronidazole, ultraviolet or ionizing radiation.²⁸ In addition, the inventors found some additional novel antibodies that were inversely associated with IM. HypB is a GTPase with a key role in nickel homeostasis.²⁹ H. pylori hypB-deficient mutant exhibits significantly decreased urease activity as there is a physical interaction between urease and HP0900/HypB.³⁰ HpaA is a lipoprotein in the flagellar sheath and outer membrane and plays an essential role in the adhesion and colonization of the gastric mucosa.³¹ Although poorly characterized, HP0709 encodes an enzyme that is involved in either DNA methylation or synthesis of some branched amino acids. HP0385 is a hypothetical protein with an unknown function. The findings for HpaA or RecA are inconsistent with a previous study showing a positive association of these two proteins with IM.¹² Considering the dual faceted roles of these antigens, along with their various parts in coordination with other antigens and the external environment, further studies should investigate the various host-antigen interactions of these proteins. It is also possible that these proteins are important for colonization and establishment of successful infection but less relevant when the microenvironment changes due to hypochlorhydria and other tissue transformations related to atrophy.

Extensive IM confers a higher risk of gastric cancer. This exploratory case-case analysis identified three antibody associations (anti-HP0516/HslU, anti-HP0385 and anti-HP1453/homD) with antral restricted IM (vs. corpus extension). The corresponding bacterial antigens have not been well studied. In particular, homD is a conserved gene and little is known about its encoding OMP (HP1453/homD).³² Further studies are warranted to address the potential differences in the humoral responses to H. pylori by gastric IM anatomical subsite.

Although H. pylori infection can be detected by many methods, their sensitivity and specificity are variable. Taken together, the results of four different H. pylori tests assessing current and past infections, the high-risk population in Chile seem to have had an almost universal bacterial exposure. There are no specific bacterial proteins that are recognized in all H. pylori-infected individuals and do not show correlations with other proteins. Differential humoral responses to specific H. pylori antigens could be related to variable protein expression influenced by the stomach microenvironment. Loh et al.,³³ have documented in rodent models increased expression and translocation of CagA in response to high salt conditions. Noto et al.,³⁴ reported a similar increased expression of CagA in strains growing under iron-limiting conditions. Anti-GroEL may be a suitable biomarker of either current or past infection. The NAPPA anti-GroEL fairly agreed with a commercial wcELISA. The inventors identified 16 antibodies as markers of current H. pylori infection, including some novel candidates. In currently infected individuals, flagellar antigens exhibit seropositivity as high as 90%.³⁵ The NAPPA array included a total of 28 flagellar antigens and found positive associations with HP0870/flgE, HP0601/flaA, HP0295/fla that showed overall prevalences ranged from 40% to 58% in all study individuals, and from 50% to 71% in currently infected individuals. Based on in vitro studies, HP1118/gGT seems to be a pathogenic factor associated with H. pylori-induced peptic ulcer disease.³⁶ As mentioned above, Pan et al., reported an inverse IM association with anti-HP1118-gGT.¹¹ Similarly, the inventors found a suggestive association in this study and a significant inverse association in the previous NAPPA gastric cancer study.⁵ To the inventors' knowledge, no data is available on the functions of anti-HP1110. Future research may explore the role of these antigens in vaccine development.

To date, there are two bacterial Genome-Wide Association Studies (GWAS) of gastric cancer. Berthenet et al., identified 32 significant loci (HP0068, HP0102, HP0269, HP0290, HP0468, HP0524, HP0527, HP0528, HP0531, HP0532, HP0540, HP0541, HP0544, HP0555, HP0569, HP0615, HP0709, HP0747, HP0797, HP0906, HP0936, HP1004, HP1046, HP1055, HP1149, HP1177, HP1184, HP1243, HP1331, HP1421, HP1460, HP1572) by comparing hpEurope genomes from patients with gastric cancer (n=49) with genomes from patients with gastritis (non-atrophic and atrophic with and without IM; n=124).³⁷ On the other hand, Tuan and Yahara et al.³⁸ identified 15 significant loci (HP0004, HP0082, HP0096, HP0130, HP0231, HP0463, HP0490, HP0547, HP0776, HP0807, HP0843, HP1250, HP1440, HP1467) by comparing hspEAsia genomes from patients with gastric cancer (n=125) and genomes from patients with duodenal ulcer (n=115). Likely due to differences in the control group (gastritis vs. duodenal ulcer) and the underlying genetic structure of hpEurope and hspEAsia populations, there are no common hits between the GWAS. Notably, two of the candidate antibodies, anti-HP1177/Omp27/HopQ (positive association with IM) and anti-HP0709 (inverse association with IM and positive association with current H. pylori infection) represent gastric cancer hits in the hpEurope GWAS. HP1177/Omp27/HopQ was identified under the comparison including IM in the case group, while HP0709 was identified under the comparison including IM in the control group. Two of the additional candidate antibodies, anti-HP0547/CagA (positive association with IM) and anti-HP0231 (positive association with current H. pylori infection), represent gastric cancer hits in the hspEAsia GWAS.

The strengths of the serology IM study include the use of a well-validated and highly reproducible state-of-the-art microarray technology. Secondly, the inventors employed a two-stage approach with discovery and independent validation using blinded testing to ensure the rigor of the findings. Importantly, the inventors tested well-characterized samples with a high prevalence of H. pylori infection and data from several complementary tests documenting current and past H. pylori infections.

Antibodies to several specific H. pylori proteins are associated with modest gastric cancer risk in prospective cohort studies.^{39, 43} To date, none of the proposed antibodies have enough discriminatory power to distinguish gastric cancer patients from cancer-free individuals for screening. The data herein suggest a moderate gain in discriminating IM and NAG with a combined IgG antibody model compared to an anti-CagA only model. Additional candidate anti-OMP antibodies could be tested in pre-diagnostic samples.

Besides the etiological importance of the identified H. pylori antibodies in gastric

carcinogenesis, a non-invasive blood test for IM based on the antibodies may have a potential translation to noninvasive detection of patients with this premalignant lesion. After additional validation, the candidate antibodies in combination with other serologic biomarkers (e.g., pepsinogens), could have a direct clinical application for triaging high-risk individuals for further diagnostic procedures, particularly in places where mass gastric cancer screening resources are limited.

Conclusion: This study represents the most comprehensive assessment of anti-H. pylori antibody profiles in IM. The target antigens for these novel antibodies may act together with CagA in the progression to IM. Along with other biomarkers, specific H. pylori antibodies may identify IM patients, who would benefit from surveillance.

### CITED REFERENCES

1. Correa P. Human gastric carcinogenesis: a multistep and multifactorial process—First American Cancer Society Award Lecture on Cancer Epidemiology and Prevention. Cancer Research 1992;52:6735-40.
2. Kokkola A, Kosunen T U, Puolakkainen P, et al. Spontaneous disappearance of Helicobacter pylori antibodies in patients with advanced atrophic corpus gastritis. APMIS 2003;111:619-24.
3. Spence A D, Cardwell C R, McMenamin U C, et al. Adenocarcinoma risk in gastric atrophy and intestinal metaplasia: a systematic review. BMC Gastroenterol 2017;17:157.
4. Dinis-Ribeiro M, Areia M, de Vries A C, et al. Management of precancerous conditions and lesions in the stomach (MAPS): guideline from the European Society of Gastrointestinal Endoscopy (ESGE), European Helicobacter Study Group (EHSG), European Society of Pathology (ESP), and the Sociedade Portuguesa de Endoscopia Digestiva (SPED). Endoscopy 2012;44:74-94.
5. Song L, Song M, Rabkin C S, et al. Helicobacter pylori Immunoproteomic Profiles in Gastric Cancer. J Proteome Res 2021;20:409-419.
6. Koelblen T, Berge C, Cherrier M V, et al. Molecular dissection of protein-protein interactions between integrin alpha5beta1 and the Helicobacter pylori Cag type IV secretion system. FEBS J 2017;284:4143-4157.
7. Song L S, Wallstrom G, Yu X B, et al. Identification of Antibody Targets for Tuberculosis Serology using High-Density Nucleic Acid Programmable Protein Arrays. Molecular & Cellular Proteomics 2017;16:S277-S289.
8. Mejias-Luque R, Gerhard M. Immune Evasion Strategies and Persistence of Helicobacter pylori. Curr Top Microbiol Immunol 2017;400:53-71.
9. Veijola L, Oksanen A, Sipponen P, et al. Evaluation of a Commercial Immunoblot, Helicoblot 2.1, for Diagnosis of Helicobacter pylori Infection. Clinical and Vaccine Immunology 2008;15:1705-1710.
10. Murata-Kamiya N. Pathophysiological functions of the CagA oncoprotein during infection by Helicobacter pylori. Microbes Infect 2011;13:799-807.
11. Pan K F, Formichella L, Zhang L, et al. Helicobacter pylori antibody responses and evolution of precancerous gastric lesions in a Chinese population. Int J Cancer 2014;134:2118-25.
12. Epplein M, Butt J, Zhang Y, et al. Validation of a Blood Biomarker for Identification of Individuals at High Risk for Gastric Cancer. Cancer Epidemiology Biomarkers & Prevention 2018;27:1472-1479.
13. Xu C, Soyfoo D M, Wu Y, et al. Virulence of Helicobacter pylori outer membrane proteins: an updated review. Eur J Clin Microbiol Infect Dis 2020;39:1821-1830.
14. Javaheri A, Kruse T, Moonens K, et al. Helicobacter pylori adhesin HopQ engages in a virulence-enhancing interaction with human CEACAMs. Nat Microbiol 2016;2:16189.
15. Xia R, Zhang B, Wang X, et al. Pathogenic interactions between Helicobacter pylori adhesion protein HopQ and human cell surface adhesion molecules CEACAMs in gastric epithelial cells. Iran J Basic Med Sci 2019;22:710-715.
16. Dooyema S D R, Krishna U S, Loh J T, et al. Helicobacter pylori-Induced TLR9 Activation and Injury Are Associated With the Virulence-Associated Adhesin HopQ. J Infect Dis 2021;224:360-365.
17. Taxauer K, Hamway Y, Ralser A, et al. Engagement of CEACAM1 by Helicobacter pylori HopQ Is Important for the Activation of Non-Canonical NF-kappaB in Gastric Epithelial Cells. Microorganisms 2021;9.
18. Shan Y, Lu X, Han Y, et al. Helicobacter pylori Outer Membrane Protein 18 (Hp1125) Is Involved in Persistent Colonization by Evading Interferon-gamma Signaling. Biomed Res Int 2015;2015:571280.
19. Huang J Y, Sweeney E G, Sigal M, et al. Chemodetection and Destruction of Host Urea Allows Helicobacter pylori to Locate the Epithelium. Cell Host Microbe 2015;18:147-56.
20. Goers Sweeney E, Henderson J N, Goers J, et al. Structure and proposed mechanism for the pH-sensing Helicobacter pylori chemoreceptor TlpB. Structure 2012;20:1177-88.
21. Pernitzsch S R, Alzheimer M, Bremer B U, et al. Small RNA mediated gradual control of lipopolysaccharide biosynthesis affects antibiotic resistance in Helicobacter pylori. Nat Commun 2021;12:4433.
22. Suganuma M, Kurusu M, Suzuki K, et al. New tumor necrosis factor-alpha-inducing protein released from Helicobacter pylori for gastric cancer progression. J Cancer Res Clin Oncol 2005;131:305-13.
23. Jang J Y, Yoon H J, Yoon J Y, et al. Crystal structure of the TNF-alpha-Inducing protein (Tipalpha) from Helicobacter pylori: Insights into Its DNA-binding activity. J Mol Biol 2009;392:191-7.
24. Voland P, Weeks D L, Vaira D, et al. Specific identification of three low molecular weight membrane-associated antigens of Helicobacter pylori. Aliment Pharmacol Ther 2002;16:533-44.
25. Jung J, Lee C J, Jeon Y H, et al. Solution structure and backbone dynamics of the biotinylation domain of Helicobacter pylori biotin-carboxyl carrier protein. Bulletin of the Korean Chemical Society 2008;29:347-351.
26. Wang G, Hong Y, Olczak A, et al. Dual Roles of Helicobacter pylori NapA in inducing and combating oxidative stress. Infect Immun 2006;74:6839-46.
27. Zhao Y, Cai Y, Chen Z, et al. SpoT-mediated NapA upregulation promotes oxidative stress-induced Helicobacter pylori biofilm formation and confers multidrug resistance. Antimicrob Agents Chemother 2021.
28. Orillard E, Radicella J P, Marsin S. Biochemical and cellular characterization of Helicobacter pylori RecA, a protein with high-level constitutive expression. J Bacteriol 2011;193:6490-7.
29. Maroney M J, Ciurli S. Nickel as a virulence factor in the Class I bacterial carcinogen, Helicobacter pylori. Semin Cancer Biol 2021.
30. Olson J W, Mehta N S, Maier R J. Requirement of nickel metabolism proteins HypA and HypB for full activity of both hydrogenase and urease in Helicobacter pylori. Mol Microbiol 2001;39:176-82.
31. Banga Ndzouboukou J L, Lei Q, Ullah N, et al. Helicobacter pylori adhesins: HpaA a potential antigen in experimental vaccines for H. pylori. Helicobacter 2021;26:e12758.
32. Servetas S L, Kim A, Su H, et al. Comparative analysis of the Hom family of outer membrane proteins in isolates from two geographically distinct regions: The United States and South Korea. Helicobacter 2018;23:e12461.
33. Loh J T, Tones V J, Cover T L. Regulation of Helicobacter pylori cagA expression in response to salt. Cancer Res 2007;67:4709-15.
34. Noto J M, Gaddy J A, Lee J Y, et al. Iron deficiency accelerates Helicobacter pylori-induced carcinogenesis in rodents and humans. J Clin Invest 2013;123:479-92.
35. Khalifeh Gholi M, Kalali B, Formichella L, et al. Helicobacter pylori FliD protein is a highly sensitive and specific marker for serologic diagnosis of H. pylori infection. Int J Med Microbiol 2013;303:618-23.
36. Zhang X, Zhang J, Yang F, et al. Immunization with Heat Shock Protein A and gamma-Glutamyl Transpeptidase Induces Reduction on the Helicobacter pylori Colonization in Mice. PLoS One 2015;10:e0130391.
37. Berthenet E, Yahara K, Thorell K, et al. A GWAS on Helicobacter pylori strains points to genetic variants associated with gastric cancer risk. BMC Biol 2018;16:84.
38. Tuan V P, Yahara K, Dung H D Q, et al. Genome-wide association study of gastric cancer- and duodenal ulcer-derived Helicobacter pylori strains reveals discriminatory genetic variations and novel oncoprotein candidates. Microb Genom 2021;7.
39. Cai H, Ye F, Michel A, et al. Helicobacter pylori blood biomarker for gastric cancer risk in East Asia. Int J Epidemiol 2016;45:774-81.
40. Shakeri R, Malekzadeh R, Nasrollahzadeh D, et al. Multiplex H. pylori Serology and Risk of Gastric Cardia and Noncardia Adenocarcinomas. Cancer Res 2015;75:4876-83.
41. Camargo M C, Beltran M, Conde-Glez C J, et al. Serological response to Helicobacter pylori infection among Latin American populations with contrasting risks of gastric cancer. Int J Cancer 2015;137:3000-5.
42. Gisbert J P, Pajares J M. Review article: 13C-urea breath test in the diagnosis of Helicobacter pylori infection—a critical review. Aliment Pharmacol Ther 2004;20:1001-17.
43. Gao L, Weck M N, Michel A, et al. Association between Chronic Atrophic Gastritis and Serum Antibodies to 15 Helicobacter pylori Proteins Measured by Multiplex Serology. Cancer Research 2009;69:2973-2980.
44. WO2021062041.

TABLE 8
Sequences

| SEQ ID | |
|---|---|
| NO. | DNA Sequence |
| SEQ ID | ATGATGTTTTCTTCAATGTTTGCTTCGTTGGGGACTCGTATCATGCTGGTCG |
| NO: 1 | TGTTAGCCGCTCTTTTAGGTTTAGGGGGGCTTTTTATTGGTTTTGTAAAGGT |
| | TATGCAAAAAGATGTGTTAGCGCAACTCATGGAGCATTTAGAAACCGGGC |
| | AATACAAAAAGCGTGAAAAAACGCTCGCTTACATGACAAAAATTATTGAA |
| | CAGGGCATTCATGAGTATTACAAAAATTTTGACAATGCTACTGCAAGAAA |
| | AATGGCGTTAGATTATTTCAAACGCATCAACGACGATAAGGGCATGATTT |
| | ATATGGTGGTGGTGGATAAAAACGGGGTGGTATTGTTTGATCCGGTCAAT |
| | CCTAAAACCGTAGGCCAATCAGGGCTTGACGCTCAGAGCGTTGATGGGGT |
| | GTATTATGTTAGGGGGTATTTGGAGGCGGCCAAAAAAGGGGGAGGCTACA |
| | CTTATTATAAAATGCCTAAATACGATGGAGGCGTACCGGAGAAAAAATTC |
| | GCCTACTCGCATTATGATGAAGTTTCTCAAATGGTGATCGCAACGACTTCC |
| | TATTACACTGACATTAACACAGAAAATAAAGCGATCAAAGAAGGCGTGAA |
| | TAAGGTTTTTGATGAAAACACCACGAAATTATTCCTTTGGATACTGACAGC |
| | GACGATAGCGCTAGTGGTTTTGACGCTCATATACGCTAAATTAAGGATCGT |
| | GAAACGCATTGATGAACTGGTCCTTAAAATCAACGCTTTTAGCCGTGGGG |
| | ATAAGGATTTGAGAGCCAAAATTGATGTGGGTGATCGCAACGATGAAATC |
| | TCGCAAGTGGGCCGTGGGATCAATTTGTTTGTGGAAAACGCCCGCTTGATT |
| | ATGGAAGAGATTAAAGGGATTTCCACCCTCAATAAAACTTCAATGGATAA |
| | ATTAGTCCAAATCACGCAAGAAACCCAAAAGAGCATGAAAGATTCCTCAA |
| | CCACCCTAAATTCCGTGAAAAATAAAGCCACTGATATAGCGAGCATGATG |
| | AATGCTTCCATAGAGCAATCTCAAGGGTTAAGGAAGCGTTTGATTGAAAC |
| | GCAAGGGCTGGTCAAAGAGAGCAAGGATGCGATCGGGGATTTATTTTCTC |
| | AAATCACAGAGAGCGCGCACACTGAAGAGGAACTCTCTAGCAAAGTGGA |
| | GCAGCTAAGCCGTAACGCTGATGATGTCAAATCCATTCTGGATATTATCAA |
| | TGATATTGCCGATCAAACGAATTTATTAGCCCTAAACGCTGCTATTGAAGC |
| | CGCAAGGGCTGGCGAGCATGGCAGAGGCTTTGCGGTGGTGGCTGATGAAG |
| | TTAGGAATTTAGCCGGGCGCACTCAAAAGTCTTTAGCCGAAATCAATTCCA |
| | CTATCATGGTGATTGTCCAAGAAATCAATGCCGTGAGTTCGCAAATGAATC |
| | TCAATTCGCAAAAAATGGAGCGTTTGAGCGATATGAGTAAAAGCGTGCAA |
| | GAAACTTACGAAAAAATGAGTTCTAATTTAAGCTCAGTCGTGTCAGACAG |
| | CAATCAAAGCATGGACGATTACGCCAAATCCGGACACCAAATTGAAGTTA |
| | TGGTAAGCGATTTTGCAGAGGTGGAAAAAGTGGCTTCTAAGACTTTAGCG |
| | GATTCTTCAGATATTTTAAACATCGCTACGCATGTGAGTGGAACGACCATG |
| | AATTTAGACAAACAAGTGAATTTGTTTAAAACT |
| SEQ ID | ATGGCAATAGATGAAGACAAACAAAAAGCGATTTCTTTAGCGATCAAACA |
| NO: 2 | AATTGATAAGGTTTTTGGTAAGGGGGCGTTGGTGCGCCTTGGGGATAAGC |
| | AAGTAGAAAAGATTGACTCTATTTCTACAGGCTCGTTAGGGTTGGATCTGG |
| | CTTTAGGGATTGGGGGCGTTCCAAAGGGTAGGATCATTGAAATTTATGGG |
| | CCAGAGTCAAGTGGGAAGACCACTCTAAGCTTGCATATTATTGCAGAATG |
| | CCAAAAAAATGGCGGCGTGTGCGCGTTCATTGACGCTGAGCATGCCCTAG |
| | ATGTGCATTACGCTAAGAGATTGGGCGTGGATACGGAAAATCTACTCGTTT |
| | CCCAACCTGATACAGGCGAGCAAGCTTTAGAGATTTTAGAAACGATCACC |
| | AGAAGCGGAGGGATTGATTTAGTGGTGGTGGATTCTGTGGCGGCTCTTAC |
| | GCCTAAAGCGGAGATTGATGGGGATATGGGCGATCAGCATGTGGGCTTGC |
| | AAGCAAGGCTTATGAGCCATGCGTTAAGAAAAATCACCGGTGTCTTGCAC |
| | AAGATGAACACTACTCTCATTTTTATCAATCAAATCAGGATGAAGATTGGC |
| | ATGATGGGTTATGGGAGTCCAGAGACCACAACCGGAGGTAACGCCTTAAA |
| | ATTCTATGCGAGCGTTAGGATTGATATTAGAAGGATTGCCTCCCTCAAACA |
| | AAACGAACAGCATATCGGTAATAGGGCTAAAGCCAAAGTGGTTAAAAATA |
| | AAGTCGCTCCGCCTTTTAGAGAAGCGGAATTTGACATCATGTTTGGGGAA |
| | GGGATTTCTAAAGAGGGCGAAATCATTGATTACGGTGTGAAATTAGACAT |
| | TGTGGATAAGAGTGGGGCATGGCTTAGCTACCAGGATAAAAAGCTAGGGC |
| | AAGGCAGAGAAAACGCTAAAGCCTTACTGAAAGAAGACAAAGCCCTAGC |
| | GGATGAAATCACTCTTAAGATTAAAGAGAGTATTGGCTCTAATGAAGAGA |
| | TCATGCCCTTACCGGATGAGCCTTTAGAAGAAATGGAA |
| SEQ ID | ATGAAAACATTTGAAATTCTAAAACATTTGCAAGCGGATGCGATCGTGTT |
| NO: 3 | ATTTATGAAAGTGCATAACTTCCATTGGAATGTGAAAGGCACCGATTTTTT |
| | CAATGTGCATAAAGCCACTGAAGAAATTTATGAAGAGTTTGCGGACATGT |
| | TTGACGATCTCGCTGAAAGGATCGTTCAATTAGGGCATCACCCCTTAGTCA |
| | CTTTATCCGAAGCGATCAAACTCACTCGTGTTAAAGAAGAAACTAAAACG |
| | AGCTTCCACTCTAAAGACATCTTTAAAGAAATTCTAGAGGACTACAAATAT |
| | CTAGAAAAAGAATTTAAAGAGCTCTCTAACACCGCTGAAAAAGAAGGCGA |
| | TAAAGTTACCGTAACTTATGCGGATGATCAATTAGCCAAGTTGCAAAAAT |
| | CCATTTGGATGCTGCAAGCCCATTTGGCT |
| SEQ ID | ATGAACCTTTCTGAAATTGAAGAGTTGATCAAAGAATTTAAAGCTTCTGAT |
| NO: 4 | TTGGGGCATTTGAAATTAAAGCATGAGCATTTTGAGTTGGTTTTGGATAAA |
| | GAATCCGCTTATGCGAAAAAAAGTGCGTTAAATCCCGCCCATTCTCCAGCC |
| | CCCATTATGGTAGAAGCGAGCATGCCAAGCGTCCAAACCCCTGTGCCTAT |
| | GGTATGCACCCCTATTGTGGATAAAAAAGAAGATTTCGTGCTTTCGCCTAT |
| | GGTAGGCACTTTTTATCATGCACCCTCCCCTGGGGCTGAGCCTTATGTCAA |
| | AGCGGGCGATACGCTTAAAAAAGGGCAAATCGTGGGCATTGTAGAAGCG |
| | ATGAAAATCATGAATGAAATTGAAGTGGAATACCCTTGCAAGGTGGTTTC |
| | TGTTGAAGTGGGAGACGCTCAACCGGTAGAATACGGCACAAAACTCATCA |
| | AAGTTGAAAAGCTT |
| SEQ ID | ATGATGCAATCTTTAAGTTTATTAAACCAATTGGGCGCTAAAATTGATGAA |
| NO: 5 | TTGATTGAAAAAATCAAAAAACAAGAAGAAGAGTTGAACGCTTTACGCCA |
| | AGCAAACACCACCCTGAATGCGCAAAATGAAGAAAAAGACATTCAAATC |
| | GCTATTTTATACGATGAATTGAGCGCTAAAGATAAGGGTATTCAAGGTCTT |
| | TATGACAAAATCTCTGATTTGTTGTCA |
| SEQ ID | ATGACTAACGAAACTATTGACCAACAACCACAAACCGAAGCGGCTTTTAA |
| NO: 6 | CCCGCAGCAATTTATCAACAATCTTCAAGTGGCTTTTCTTAAAGTTGATAA |
| | CGCTGTCGCTTCATACGATCCTGATCAAAAACCAATCGTTGATAAGAATGA |
| | TAGGGATAACAGGCAAGCTTTTGATGGAATCTCGCAATTAAGGGAAGAAT |
| | ACTCCAATAAAGCGATCAAAAATCCTACCAAAAAGAATCAGTATTTTTCA |
| | GACTTTATCAATAAGAGCAATGATTTAATCAACAAAGACAATCTCATTGAT |
| | ATAGGTTCTTCCATAAAAAGCTTTCAGAAATTTGGGACTCAGCGTTACCGA |
| | ATTTTCACAAGTTGGGTGTCCCATCAAAACGATCCGTCTAAAATCAACACC |
| | CGATCGATCCGAAATTTTATGGAAAATATCATACAACCCCCTATCCCTGAT |
| | GACAAAGAAAAAGCAGAGTTTTTGAAATCTGCCAAACAATCTTTTGCAGG |
| | AATCATTATAGGGAATCAAATCCGAACGGATCAAAAGTTCATGGGCGTGT |
| | TTGATGAATTCTTGAAAGAAAGGCAAGAAGCAGAAAAAAATGGAGAGCC |
| | TACTGGTGGGGATTGGTTGGATATTTTTTTATCATTTGTATTTAACAAAGA |
| | ACAATCTTCTGATGTCAAAGAAGCAATCAATCAAGAACCAGTTCCCCATG |
| | TCCAACCAGATATAGCCACTACCACCACCCACATACAAGGCTTACCGCCT |
| | GAATCTAGGGATTTGCTTGATGAAAGGGGTAATTTTTCTAAATTCACTCTT |
| | GGCGATATGGAAATGTTAGATGTTGAGGGCGTCGCCGACATTGATCCTAA |
| | TTACAAGTTCAATCAATTATTGATTCACAATAACGCTCTGTCTTCTGTGTTA |
| | ATGGGGAGTCATAATGGCATAGAACCTGAAAAAGTTTCATTATTGTATGC |
| | GGGCAATGGTGGTTTTGGAGCCAAGCACGATTGGAACGCCACCGTTGGTT |
| | ATAAAAACCAACAAGGCGACAATGTGGCTACACTCATTAATGTGCATATG |
| | AAAAACGGCAGTGGCTTAGTCATAGCAGGTGGTGAGAAAGGGATTAACA |
| | ACCCTAGTTTTTATCTCTACAAAGAAGACCAACTCACAGGCTCACAACGA |
| | GCATTGAGTCAAGAAGAGATCCGAAACAAAATAGATTTCATGGAATTTCT |
| | TGCACAAAACAATGCTAAATTAGACAACTTGAGCGAGAAAGAGAAAGAA |
| | AAATTCCAAAATGAGATTGAAGATTTTCAAAAAGACTCTAAGGCTTATTTA |
| | GACGCCCTAGGGAATGATCGTATTGCCTTTGTTTCTAAAAAAGACCCAAA |
| | ACATTCAGCTTTAATTACTGAGTTTGGTAAGGGGGATTTGAGCTACACTCT |
| | CAAAGATTATGGGAAAAAAGCAGATAGAGCTTTAGATAGGGAGAAAAAT |
| | GTTACTCTTCAAGGTAACCTAAAACATGATAGCGTGATGTTTGTTAATTAT |
| | TCTAATTTCAAATACACCAACGCCTCCAAGAGTCCTGATAAGGGTGTAGG |
| | CGTTACAAATGGCGTTTCCCATTTAGACGCAGGCTTTAGCAAGGTAGCTGT |
| | CTTTAATTTGCCTGATTTAAATAATCTCGCTATCACTAGTTTCGTAAGGCG |
| | GAATTTAGAGAATAAACTAGTCACTGAAGGATTGTCCCTACAAGAAGCTA |
| | ATAAGCTTATCAAAGATTTTTTGAGCAGCAACAAAGAATTGGTTGGAAAA |
| | GCTTTAAACTTCAATAAAGCTGTAGCTGACGCTAAAAACACAGGCAACTA |
| | TGATGAAGTGAAAAAAGCTCAGAAAGATCTTGAAAAATCTCTAAGGAAAC |
| | GAGAGCATTTAGAGAAAGAAGTAGAGAAAAAATTGGAGAGCAAAAGCGG |
| | CAACAAAAATAAAATGGAAGCGAAAGCTCAAGCTAACAGCCAAAAAGAT |
| | AAGATTTTTGCGTTGATCAATAAAGAGGCTAATAGGGACGCAAGAGCAAT |
| | CGCTTACTCTCAGAATCTTAAAGGCATCAAAAGGGAATTGTCTGATAAACT |
| | TGAAAAAATCAACAAGGATTTGAAAGACTTTAGTAAATCTTTTGATGAATT |
| | CAAAAATGGCAAAAATAAGGATTTTAGCAAGGCAGAAGAAACGCTAAAA |
| | GCCCTTAAAGGCTCGGTGAAAGATTTAGGTATCAATCCGGAATGGATTTC |
| | AAAAGTTGAAAACCTTAATGCAGCTTTGAATGAATTCAAAAATGGCAAAA |
| | ATAAGGATTTCAGCAAGGTAACGCAAGCAAAAAGCGACCTTGAAAATTCC |
| | GTTAAAGATGTGATCATCAATCAAAAGATAACGGATAAAGTTGACAATCT |
| | CAATCAAGCGGTATCAATGGCTAAAGCAACGGGTGATTTCAGTAGGGTAG |
| | AGCAAGCGTTAGCCGATCTCAAAAACTTCTCAAAGGAGCAATTGGCTCAA |
| | CAAACTCAAAAAAATGAAAGTTTCAATGTTGGAAAAAAATCTGAAATATA |
| | TCAATCCGTTAAGAATGGTGTGAACGGAACCCTAGTCGGTAATGGGTTAT |
| | CTGGAATAGAGGCCACAGCTCTCGCCAAAAATTTTTCGGATATCAAGAAA |
| | GAATTGAATGAGAAATTTAAAAATTTCAATAACAATAACAATAATGGACT |
| | CGAAAACGAACCCATTTATGCTAAAGTTAATAAAAAGAAAACAGGACAA |
| | GTAGCTAGCCCTGAAGAACCCATTTACGCTCAAGTTGCTAAAAAGGTGAA |
| | TGCAAAAATTGACCGACTCAATCAAGCAGCAAGTGGTTTGGGTGGTGTAG |
| | GGCAAGCGGGCTTCCCTTTGAAAAGGCATGATAAAGTTGATGATCTCAGT |
| | AAGGTAGGGCGATCAGTTAGCCCTGAACCCATTTATGCTACGATTGATGAT |
| | CTCGGCGGACCTTTCCCTTTGAAAAGGCATGATAAAGTTGATGATCTCAGT |
| | AAGGTAGGGCGATCAGTTAGCCCTGAACCCATTTATGCTACGATTGATGAT |
| | CTCGGCGGACCTTTCCCTTTGAAAAGGCATGATAAAGTTGATGATCTCAGT |
| | AAGGTAGGGCGATCAGTTAGCCCTGAACCCATTTATGCTACGATTGATGAT |
| | CTCGGCGGACCTTTCCCTTTGAAAAGGCATGATAAAGTTGATGATCTCAGT |
| | AAGGTAGGGCTTTCAAGGAATCAAGAATTGGCTCAGAAAATTGACAATCT |
| | CAGTCAAGCGGTATCAGAAGCTAAAGCAGGTTTTTTTAGCAATCTAGAGC |
| | AAACGATAGACAAGCTCAAAGATTCTACAAAATACAATTCTGTGAATCTA |
| | TGGGTTGAAAGTGCAAAAAAAGTGCCTGCTAGTTTGTCAGCGAAACTAGA |
| | CAATTACGCTACTAACAGCCACACACGCATTAATAGCAATATCCAAAATG |
| | GAGCAATCAATGAAAAAGCGACCGGTATGCTAACGCAAAAAAACCCTGA |
| | GTGGCTCAAGCTCGTGAATGATAAGATAGTTGCGCATAATGTGGGAAGCG |
| | TTCCTTTGTCAGAGTATGATAAAATTGGCTTCAACCAGAAGAATATGAAA |
| | GATTATTCTGATTCGTTCAAGTTTTCCACCAAGTTGAACAATGCTGTAAAA |
| | GACGTTAAGTCTAGCTTTACGCAATTTTTAGCCAATGCATTTTCTACAGGA |
| | TATTACTCCTTGGCGAGGGAAAATGCGGAGCATGGAATCAAAAATGTTAA |
| | TACAAAAGGTGGTTTCCAAAAATCTTAA |
| SEQ ID | ATGTTAGAAAAATCTTTTTTAAAAAGCAAGCAATTATTTTTATGCGGACTG |
| NO: 7 | GGTGTTTTGATGCTGCAGGCTTGCACTTGCCCAAACACTTCACAAAGGAAT |
| | TCTTTCTTGCAAGATGTGCCTTATTGGATGTTGCAAAATCGCAGTGAGTAT |
| | ATCACGCAAGGGGTGGATAGCTCGCACATTGTAGATGGTAAGAAAACTGA |
| | AGAGATAGAAAAAATCGCTACCAAAAGAGCGACAATAAGAGTGGCACAA |
| | AATATTGTGCATAAACTTAAAGAAGCTTACCTTTCCAAAACCAATCGCATC |
| | AAGCAAAAGATCACTAATGAAATGTTTATCCAAATGACACAGCCCATTTA |
| | TGACAGCTTGATGAATGTGGATCGTTTAGGGATTTATATCAATCCTAACAA |
| | TGAGGAAGTGTTTGCGTTAGTGCGCGCGCGTGGTTTTGATAAGGACGCTTT |
| | GAGCGAAGGGTTGCATAAAATGTCCTTAGACAATCAAGCGGTGAGTATCC |
| | TTGTGGCTAAAGTGGAAGAAATCTTTAAAGATTCTGTCAATTACGGAGAT |
| | GTTAAAGTCCCTATAGCCATG |
| SEQ ID | ATGAGAAAAACGATTTCAGCGTTGTTTTTATCAGCGTGCATAGGGTTATCG |
| NO: 8 | TCTGTTTATGCAGATAACGCTTTGATTTTGCAAACCGATTTTAGTCTAAAA |
| | GATGGGGCCGTCTCGGCGATGAAAGGCGTCGCTTTCAGCGTTGATTCCCAT |
| | CTTAAAATCTTTGATTTAACGCACGAAATCCCCCCGTATAACATCTGGGAA |
| | GGCGCTTACCGCTTGTATCAGACCGCCAGTTATTGGCCAAAAGGTTCGGTA |
| | TTTGTGAGCGTAGTTGATCCGGGCGTAGGCACTAAGCGTAAATCGGTGGT |
| | ACTAAAAACTAAAAACGGCCAGTATTTCGTCTCGCCGGATAACGGCACGC |
| | TGACTTTGGTGGCACAAACTTTGGGGATTGATAGCGTGCGTGAAATTGATG |
| | AAAAAGCTAACCGCTTGAAAGGTTCTGAAAAATCCTATACTTTCCATGGTC |
| | GTGATGTGTATGCTTACACCGGTGCACGCTTGGCTTCTGGGGCGATCACAT |
| | TCGAGCAGGTCGGGCCAGAGCTTCCCCCAAAAGTCGTTGAAATTCCTTACC |
| | AAAAAGCGAAAGCCACAAAAGGGGAAGTGAAAGGTAATATCCCGATTTT |
| | GGATATTCAATATGGCAATGTTTGGAGCAACATCAGCGATAAATTACTCA |
| | ATCAAGCAAAAATCAAACTCAATGACACGCTGTGTGTAACGATTTTTAAA |
| | GGTTCTAAGAAACAATACGAAGGGAAAATGCCGTATGTCGCAAGCTTTGG |
| | CGATGTGCCAGAAGGCCAGCCGTTAGTTTATTTAAACAGCTTGTTAAATGT |
| | TTCCGTGGCGCTGAATAGGGATAATTTCGCGCAAAAATATCAAATCAAAT |
| | CCGGTGCTGACTGGAATATTGATATAAAGAAGTGCGCTAAG |
| SEQ ID | ATGAGCGAACAACGACAAGAATCTTTACAAAATAACCCTAATTTGAGTAA |
| NO: 9 | AAAAGATGTCAAAATCGTAGAAAAGATTTTGAGTAAGAACGACATTAAAG |
| | CCGCTGAAATGAAAGAACGCTATTTAAAAGAAGGGCTGTATGTGTTGAAT |
| | TTCATGAGTTCTCCCGGTAGCGGTAAAACCACGATGCTAGAAAATCTAGC |
| | GGATTTTAAAGACTTTAAGTTTTGCGTGGTAGAGGGCGATTTGCAAACCAA |
| | CAGAGATGCGGACAGATTGCGTAAAAAAGGCGTGAGTGCGCACCAGATC |
| | ACCACCGGCGAAGCATGCCATTTGGAAGCGAGCATGATTGAAGGGGCGTT |
| | TGATTTATTAAAAGATGAGGGAGCGTTAGAAAAAAGCGATTTTTTAATCA |
| | TTGAAAACGTGGGGAATTTGGTTTGCCCCTCAAGCTATAATCTAGGAGCG |
| | GCGATGAATATCGTTTTACTCTCCGTTCCAGAGGGCGATGATAAGGTGCTA |
| | AAATACCCTACGATGTTCATGTGCGCGGATGCGGTGATTATCAGTAAAGC |
| | GGATATGGTTGAGGTGTTTAATTTCAGGGTTTCTCAAGTCAAAGAAGACAT |
| | GCAAAAATTAAAGCCTGAAGCGCCTATTTTTTTAATGAGCTCTAAAGACCC |
| | TAAAAGTTTGGAAGATTTTAAAAATTTCCTTCTAGAAAAAAAGCGTGAAA |
| | ATTACCAGTCCACGCATTCGTTT |
| SEQ ID | ATGAAGAGATCTTCTGTATTTAGTTTCTTGGTAGCTTTTTTATTGGTAGCTG |
| NO: 10 | GCTGTAGTCATAAAATGGATAATAAGACTGTGGCCGGCGATGTGAGTGCT |
| | AAAACGGTTCAGACTGCACCTGTTACTACAGAACCAGCTCCAGAGAAAGA |
| | AGAGCCTAAACAAGAGCCAGCTCCAGTGGTTGAAGAAAAACCGGCTGTTG |
| | AGAGCGGGACTATCATCGCTTCTATTTATTTTGATTTTGACAAGTATGAAA |
| | TCAAAGAATCCGATCAAGAGACTTTAGATGAGATCGTGCAAAAAGCTAAA |
| | GAAAACCACATGCAAGTGCTTTTGGAAGGCAATACCGATGAATTTGGCTC |
| | TAGCGAATACAACCAAGCGCTTGGCGTTAAAAGGACTTTGAGCGTGAAAA |
| | ACGCTTTAGTCATTAAAGGGGTAGAAAAAGATATGATCAAAACCATCAGT |
| | TTTGGTGAAACCAAACCCAAATGCGCCCAAAAAACTAGAGAGTGTTATAA |
| | AGAAAACAGAAGAGTGGATGTCAAATTAATGAAG |
| SEQ ID | ATGAAAAAAACGAAAAAAACGATTCTGCTTTCTCTAACTCTCGCGGCGTC |
| NO: 11 | ATTGCTCCATGCTGAAGACAACGGCGTTTTTTTAAGCGTGGGTTATCAAAT |
| | CGGTGAAGCGGTTCAAAAAGTGAAAAACGCCGACAAGGTGCAAAAACTTT |
| | CAGACACTTATGAACAATTAAGCCGGCTTTTAACCAACGATAATGGCACA |
| | AACTCAAAGACAAGCGCGCAAGCGATCAACCAAGCGGTTAATAATTTGAA |
| | CGAACGCGCAAAAACTTTAGCCGGTGGGACAACCAATTCCCCTGCCTATC |
| | AAGCCACGCTTTTAGCGTTGAGATCGGTGTTAGGGCTATGGAATAGCATG |
| | GGTTATGCGGTCATATGCGGAGGTTATACCAAAAGTCCAGGCGAAAACAA |
| | TCAAAAAGATTTCCACTACACCGATGAGAATGGCAATGGCACTACAATCA |
| | ATTGCGGTGGGAGCACAAATAGTAATGGCACTCATAGTTCTAGTGGCACA |
| | AATACATTAAAAGCAGACAAAAATGTTTCTCTATCTATTGAGCAATATGA |
| | AAAAATCCATGAAGCTTATCAGATTCTTTCAAAAGCTTTAAAACAAGCCG |
| | GGCTTGCTCCTTTAAATAGCAAAGGGGAAAAGTTAGAAGCGCATGTAACC |
| | ACATCAAAACCAGAAAATAATAGTCAAACTAAAACGACAACTTCTGTTAT |
| | TGATACGACTAATGATGCGCAAAATCTTTTGACTCAAGCGCAAACGATTGT |
| | CAATACCCTTAAAGATTATTGCCCCATGTTGATAGCGAAATCTAGTAGTGA |
| | AAGTAGTGGCGCAGCTACTACAAACGCCCCTTCATGGCAAACAGCCGGTG |
| | GCGGCAAAAATTCATGTGCGACTTTTGGTGCGGAGTTTAGTGCCGCTTCAG |
| | ACATGATTAATAATGCGCAAAAAATCGTTCAAGAAACCCAACAACTCAGC |
| | GCCAACCAACCAAAAAATATCACACAACCCCATAATCTCAACCTTAACAC |
| | CCCTAGCAGTCTTACGGCTTTAGCTCAAAAAATGCTCAAAAATGCGCAATC |
| | TCAAGCAGAAATTTTAAAACTAGCCAATCAAGTGGAGAGCGATTTTAACA |
| | AACTTTCTTCAGGCCATCTTAAAGACTACATAGGGAAATGCGATGCGAGC |
| | GCTATAAGCAGTGCGAATATGACAATGCAAAATCAAAAGAACAATTGGGG |
| | GAACGGGTGTGCTGGCGTGGAAGAAACTCTGTCTTCATTAAAAACAAGTG |
| | CCGCTGATTTTAACAACCAAACGCCACAAATCAATCAAGCGCAAAACCTA |
| | GCCAACACCCTTATTCAAGAACTTGGCAACAACCCTTTTAGGAATATGGGC |
| | ATGATCGCTTCTTCAACCACGAATAACGGCGCCTTGAATGGCCTTGGGGTG |
| | CAAGTGGGTTATAAGCAATTTTTTGGGGAAAAGAAAAGATGGGGGTTAAG |
| | GTATTATGGTTTCTTTGATTACAACCACGCCTATATCAAATCCAATTTCTTT |
| | AACTCGGCTTCTGATGTGTGGACTTATGGGGTGGGCAGCGATTTATTGTTT |
| | AATTTCATCAATGATAAAAACACCAACTTTTTAGGCAAGAATAACAAGAT |
| | TTCAGTGGGATTTTTTGGAGGTATCGCCTTAGCAGGGACTTCATGGCTTAA |
| | TTCTCAATTCGTGAATTTAAAAACCATCAGCAATGTTTATAGCGCTAAAGT |
| | GAATACGGCTAACTTCCAATTTTTATTCAATTTGGGCTTGAGAACCAATCT |
| | CGCTAGACCTAAGAAAAAAGATAGTCATCATGCGGCTCAACATGGCATGG |
| | AATTGGGCGTGAAAATCCCTACCATTAACACGAATTATTATTCTTTTCTAG |
| | ACACTAAACTAGAATATCGAAGGCTTTATAGCGTGTATCTCAATTATGTGT |
| | TTGCCTAT |

The use of any and all examples provided herein, is intended merely to better illuminate the invention and does not pose a limitation on the scope of the invention unless otherwise claimed.

The cited references are incorporated by reference herein in their entireties. In the event that there is an inconsistency between a definition of a term in the specification as compared to a definition of the term in a cited reference, the term should be interpreted based on the definition in the specification.

We claim:

1. A method comprising:
(a) contacting an antibody-containing sample obtained from a subject with at least one H. pylori antigen selected from the group consisting of HP0547/CagA, HP1125/PalA, HP0596/Tipα, HP1177/Omp27, HP0103/TlpB, HP0709, HP0900/HypB, HP0371/FabE, HP0243/NapA, HP0153/RecA, and HP0385 to allow for antigen-antibody binding; and
(b) measuring the level of antigen-specific antibodies bound to the at least one antigen.

2. The method of claim 1, wherein step (a) comprises contacting the antibody-containing sample with at least three of the H. pylori antigens, and wherein step (b) comprises measuring the level of antigen-specific antibodies bound to the at least three antigens.

3. The method of claim 1, wherein step (a) comprises contacting the antibody-containing sample with at least five of the H. pylori antigens, and wherein step (b) comprises measuring the level of antigen-specific antibodies bound to each of the at least five antigens.

4. The method of claim 1, further comprising:
(c) screening the subject for gastric cancer if the level of the antigen-specific antibody bound to the at least one antigen is increased compared to a subject with non-atrophic gastritis, wherein the increased antigen is selected from the group consisting of HP0547/CagA, HP1125/PalA, HP0596/Tipα, HP1177/Omp27, and HP0103/TlpB; and/or if the at least one antigen is decreased compared to a subject with non-atrophic gastritis, wherein the decreased antigen is selected from the group consisting of HP0709, HP0900/HypB, HP0371/FabE, HP0243/NapA, HP0153/RecA, and HP0385.

5. The method of claim 4, further comprising administering to the subject an antibiotic to treat Helicobacter pylori infection if the level of the antigen-specific antibody bound to the at least one antigen is increased compared to a subject with non-atrophic gastritis, wherein the increased antigen is selected from the group consisting of HP0547/CagA, HP1125/PalA, HP0596/Tipα, HP1177/Omp27, and HP0103/TlpB; and/or if the at least one antigen is decreased compared to a subject with non-atrophic gastritis, wherein the decreased antigen is selected from the group consisting of HP0709, HP0900/HypB, HP0371/FabE, HP0243/NapA, HP0153/RecA, and HP0385.

6. The method of claim 4, further comprising administering a gastric cancer vaccine to the subject if the level of the antigen-specific antibody bound to the at least one antigen is increased compared to a subject with non-atrophic gastritis, wherein the increased antigen is selected from the group consisting of HP0547/CagA, HP1125/PalA, HP0596/Tipα, HP1177/Omp27, and HP0103/TlpB; and/or if the at least one antigen is decreased compared to a subject with non-atrophic gastritis, wherein the decreased antigen is selected from the group consisting of HP0709, HP0900/HypB, HP0371/FabE, HP0243/NapA, HP0153/RecA, and HP0385.

7. The method of claim 1, further comprising before step (b), contacting the antibody bound to the antigen with a detectable binding agent; and wherein step (b) comprises measuring the level of antigen specific antibodies using the detectable binding agent.

8. The method of claim 7, wherein the subject is human and the detectable binding agent comprises one or more anti-human IgG and/or anti-human IgA antibodies comprising a detectable label or conjugated to a detectable label.

9. The method of claim 1, wherein step (b) further comprises immobilizing antibody-antigen complexes to a solid support.

10. The method of claim 1, wherein the sample comprises at least one of blood, plasma, and serum.

11. The method of claim 1, further comprising contacting the sample with at least one antigen selected from the group consisting of HP0516/HslU, HP0385, and HP1453/HomD and measuring the level of antigen-specific antibodies in the sample, wherein increased levels of antibodies compared to a subject with corpus extension is indicative of antral restricted intestinal metaplasia.

12. A method comprising:
(a) contacting an antibody-containing sample obtained from a subject with at least one H. pylori antigen selected from the group consisting of HP1118/Ggt, HP1110/PorA, HP0870/FlgE, HP0407/BisC, HP0601/FlaA, HP1126/Tol B, HP1527/ComH, HP0492, HP1564/PlpA, HP0231, HP0295/Fla, HP1555/Tfs, HP0010/GroEL, HP0709, HP1341/TonB2, and HP0477/HopJ to allow for antigen-antibody binding;
(b) measuring the level of antigen-specific antibodies bound to the at least one antigen.

13. The method of claim 12, further comprising treating the subject with antibiotics if the level of antigen-specific antibodies bound to the at least one antigen is increased compared to non-infected or past infected controls.

14. The method of claim 12, further comprising before step (b), contacting the antibody bound to the antigen with a detectable binding agent; and wherein step (b) comprises measuring the level of antigen specific antibodies using the detectable binding agent.

15. The method of claim 14, wherein the subject is human and the detectable binding agent comprises one or more anti-human IgG and/or anti-human IgA antibodies comprising a detectable label or conjugated to a detectable label.

16. The method of claim 12, wherein step (b) comprises immobilizing antibody-antigen complexes to a solid support.

17. The method of claim 12, wherein the sample comprises at least one of blood, plasma, and serum.

18. The method of claim 12, wherein step (a) comprises contacting the antibody-containing sample with at least five of the H. pylori antigens, and wherein step (b) comprises measuring the level of antigen-specific antibodies bound to each of the at least five antigens.

19. A kit for detecting intestinal metaplasia comprising:
(a) at least three H. pylori antigen selected from the group consisting of HP1125/PalA, HP0596/Tipα, HP1177/Omp27, HP0103/TlpB, HP0709, HP0900/HypB, HP0371/FabE, HP0153/RecA, and HP0385; or
at least one H. pylori antigen selected from the group consisting of HP1118/Ggt, HP1110/PorA, HP0870/FlgE, HP0407/BisC, HP0601/FlaA, HP1126/TolB, HP1527/ComH, HP0492, HP1564/PlpA, HP0231, HP0295/Fla, HP1555/Tfs, HP0010/GroEL, HP0709, HP1341/TonB2, and HP0477/HopJ; and
(b) components for detecting antibodies capable of binding to the antigens.

20. A kit for detecting a current H. pylori infection comprising:
(a) at least one H. pylori antigen selected from the group consisting of HP1118/Ggt, HP1110/PorA, HP0870/FlgE, HP0407/BisC, HP0601/FlaA, HP1126/TolB, HP1527/ComH, HP0492, HP1564/PlpA, HP0231, HP0295/Fla, HP1555/Tfs, HP0010/GroEL, HP0709, HP1341/TonB2, and HP0477/HopJ; and
(b) components for detecting antibodies capable of binding to at least one of the antigens.


SEQUENCE LISTING (11 sequences)

SEQ ID NO: 1 - DNA, 1695 bp, synthetic construct
  source 1..1695: mol_type = other DNA
atgatgttttcttcaatgtttgcttcgttggggactcgtatcatgctggtcgtgttagccgctcttttaggtttaggggggctttttattggttttgtaaaggttatgcaaaaagatgtgttagcgcaactcatggagcatttagaaaccgggcaatacaaaaagcgtgaaaaaacgctcgcttacatgacaaaaattattgaacagggcattcatgagtattacaaaaattttgacaatgctactgcaagaaaaatggcgttagattatttcaaacgcatcaacgacgataagggcatgatttatatggtggtggtggataaaaacggggtggtattgtttgatccggtcaatcctaaaaccgtaggccaatcagggcttgacgctcagagcgttgatggggtgtattatgttagggggtatttggaggcggccaaaaaagggggaggctacacttattataaaatgcctaaatacgatggaggcgtaccggagaaaaaattcgcctactcgcattatgatgaagtttctcaaatggtgatcgcaacgacttcctattacactgacattaacacagaaaataaagcgatcaaagaaggcgtgaataaggtttttgatgaaaacaccacgaaattattcctttggatactgacagcgacgatagcgctagtggttttgacgctcatatacgctaaattaaggatcgtgaaacgcattgatgaactggtccttaaaatcaacgcttttagccgtggggataaggatttgagagccaaaattgatgtgggtgatcgcaacgatgaaatctcgcaagtgggccgtgggatcaatttgtttgtggaaaacgcccgcttgattatggaagagattaaagggatttccaccctcaataaaacttcaatggataaattagtccaaatcacgcaagaaacccaaaagagcatgaaagattcctcaaccaccctaaattccgtgaaaaataaagccactgatatagcgagcatgatgaatgcttccatagagcaatctcaagggttaaggaagcgtttgattgaaacgcaagggctggtcaaagagagcaaggatgcgatcggggatttattttctcaaatcacagagagcgcgcacactgaagaggaactctctagcaaagtggagcagctaagccgtaacgctgatgatgtcaaatccattctggatattatcaatgatattgccgatcaaacgaatttattagccctaaacgctgctattgaagccgcaagggctggcgagcatggcagaggctttgcggtggtggctgatgaagttaggaatttagccgggcgcactcaaaagtctttagccgaaatcaattccactatcatggtgattgtccaagaaatcaatgccgtgagttcgcaaatgaatctcaattcgcaaaaaatggagcgtttgagcgatatgagtaaaagcgtgcaagaaacttacgaaaaaatgagttctaatttaagctcagtcgtgtcagacagcaatcaaagcatggacgattacgccaaatccggacaccaaattgaagttatggtaagcgattttgcagaggtggaaaaagtggcttctaagactttagcggattcttcagatattttaaacatcgctacgcatgtgagtggaacgaccatgaatttagacaaacaagtgaatttgtttaaaact

SEQ ID NO: 2 - DNA, 1041 bp, synthetic construct
  source 1..1041: mol_type = other DNA
atggcaatagatgaagacaaacaaaaagcgatttctttagcgatcaaacaaattgataaggtttttggtaagggggcgttggtgcgccttggggataagcaagtagaaaagattgactctatttctacaggctcgttagggttggatctggctttagggattgggggcgttccaaagggtaggatcattgaaatttatgggccagagtcaagtgggaagaccactctaagcttgcatattattgcagaatgccaaaaaaatggcggcgtgtgcgcgttcattgacgctgagcatgccctagatgtgcattacgctaagagattgggcgtggatacggaaaatctactcgtttcccaacctgatacaggcgagcaagctttagagattttagaaacgatcaccagaagcggagggattgatttagtggtggtggattctgtggcggctcttacgcctaaagcggagattgatggggatatgggcgatcagcatgtgggcttgcaagcaaggcttatgagccatgcgttaagaaaaatcaccggtgtcttgcacaagatgaacactactctcatttttatcaatcaaatcaggatgaagattggcatgatgggttatgggagtccagagaccacaaccggaggtaacgccttaaaattctatgcgagcgttaggattgatattagaaggattgcctccctcaaacaaaacgaacagcatatcggtaatagggctaaagccaaagtggttaaaaataaagtcgctccgccttttagagaagcggaatttgacatcatgtttggggaagggatttctaaagagggcgaaatcattgattacggtgtgaaattagacattgtggataagagtggggcatggcttagctaccaggataaaaagctagggcaaggcagagaaaacgctaaagccttactgaaagaagacaaagccctagcggatgaaatcactcttaagattaaagagagtattggctctaatgaagagatcatgcccttaccggatgagcctttagaagaaatggaa

SEQ ID NO: 3 - DNA, 432 bp, synthetic construct
  source 1..432: mol_type = other DNA
atgaaaacatttgaaattctaaaacatttgcaagcggatgcgatcgtgttatttatgaaagtgcataacttccattggaatgtgaaaggcaccgattttttcaatgtgcataaagccactgaagaaatttatgaagagtttgcggacatgtttgacgatctcgctgaaaggatcgttcaattagggcatcaccccttagtcactttatccgaagcgatcaaactcactcgtgttaaagaagaaactaaaacgagcttccactctaaagacatctttaaagaaattctagaggactacaaatatctagaaaaagaatttaaagagctctctaacaccgctgaaaaagaaggcgataaagttaccgtaacttatgcggatgatcaattagccaagttgcaaaaatccatttggatgctgcaagcccatttggct

SEQ ID NO: 4 - DNA, 468 bp, synthetic construct
  source 1..468: mol_type = other DNA
atgaacctttctgaaattgaagagttgatcaaagaatttaaagcttctgatttggggcatttgaaattaaagcatgagcattttgagttggttttggataaagaatccgcttatgcgaaaaaaagtgcgttaaatcccgcccattctccagcccccattatggtagaagcgagcatgccaagcgtccaaacccctgtgcctatggtatgcacccctattgtggataaaaaagaagatttcgtgctttcgcctatggtaggcactttttatcatgcaccctcccctggggctgagccttatgtcaaagcgggcgatacgcttaaaaaagggcaaatcgtgggcattgtagaagcgatgaaaatcatgaatgaaattgaagtggaatacccttgcaaggtggtttctgttgaagtgggagacgctcaaccggtagaatacggcacaaaactcatcaaagttgaaaagctt

SEQ ID NO: 5 - DNA, 228 bp, synthetic construct
  source 1..228: mol_type = other DNA
atgatgcaatctttaagtttattaaaccaattgggcgctaaaattgatgaattgattgaaaaaatcaaaaaacaagaagaagagttgaacgctttacgccaagcaaacaccaccctgaatgcgcaaaatgaagaaaaagacattcaaatcgctattttatacgatgaattgagcgctaaagataagggtattcaaggtctttatgacaaaatctctgatttgttgtca

SEQ ID NO: 6 - DNA, 3744 bp, synthetic construct
  source 1..3744: mol_type = other DNA
atgactaacgaaactattgaccaacaaccacaaaccgaagcggcttttaacccgcagcaatttatcaacaatcttcaagtggcttttcttaaagttgataacgctgtcgcttcatacgatcctgatcaaaaaccaatcgttgataagaatgatagggataacaggcaagcttttgatggaatctcgcaattaagggaagaatactccaataaagcgatcaaaaatcctaccaaaaagaatcagtatttttcagactttatcaataagagcaatgatttaatcaacaaagacaatctcattgatataggttcttccataaaaagctttcagaaatttgggactcagcgttaccgaattttcacaagttgggtgtcccatcaaaacgatccgtctaaaatcaacacccgatcgatccgaaattttatggaaaatatcatacaaccccctatccctgatgacaaagaaaaagcagagtttttgaaatctgccaaacaatcttttgcaggaatcattatagggaatcaaatccgaacggatcaaaagttcatgggcgtgtttgatgaattcttgaaagaaaggcaagaagcagaaaaaaatggagagcctactggtggggattggttggatatttttttatcatttgtatttaacaaagaacaatcttctgatgtcaaagaagcaatcaatcaagaaccagttccccatgtccaaccagatatagccactaccaccacccacatacaaggcttaccgcctgaatctagggatttgcttgatgaaaggggtaatttttctaaattcactcttggcgatatggaaatgttagatgttgagggcgtcgccgacattgatcctaattacaagttcaatcaattattgattcacaataacgctctgtcttctgtgttaatggggagtcataatggcatagaacctgaaaaagtttcattattgtatgcgggcaatggtggttttggagccaagcacgattggaacgccaccgttggttataaaaaccaacaaggcgacaatgtggctacactcattaatgtgcatatgaaaaacggcagtggcttagtcatagcaggtggtgagaaagggattaacaaccctagtttttatctctacaaagaagaccaactcacaggctcacaacgagcattgagtcaagaagagatccgaaacaaaatagatttcatggaatttcttgcacaaaacaatgctaaattagacaacttgagcgagaaagagaaagaaaaattccaaaatgagattgaagattttcaaaaagactctaaggcttatttagacgccctagggaatgatcgtattgcctttgtttctaaaaaagacccaaaacattcagctttaattactgagtttggtaagggggatttgagctacactctcaaagattatgggaaaaaagcagatagagctttagatagggagaaaaatgttactcttcaaggtaacctaaaacatgatagcgtgatgtttgttaattattctaatttcaaatacaccaacgcctccaagagtcctgataagggtgtaggcgttacaaatggcgtttcccatttagacgcaggctttagcaaggtagctgtctttaatttgcctgatttaaataatctcgctatcactagtttcgtaaggcggaatttagagaataaactagtcactgaaggattgtccctacaagaagctaataagcttatcaaagattttttgagcagcaacaaagaattggttggaaaagctttaaacttcaataaagctgtagctgacgctaaaaacacaggcaactatgatgaagtgaaaaaagctcagaaagatcttgaaaaatctctaaggaaacgagagcatttagagaaagaagtagagaaaaaattggagagcaaaagcggcaacaaaaataaaatggaagcgaaagctcaagctaacagccaaaaagataagatttttgcgttgatcaataaagaggctaatagggacgcaagagcaatcgcttactctcagaatcttaaaggcatcaaaagggaattgtctgataaacttgaaaaaatcaacaaggatttgaaagactttagtaaatcttttgatgaattcaaaaatggcaaaaataaggattttagcaaggcagaagaaacgctaaaagcccttaaaggctcggtgaaagatttaggtatcaatccggaatggatttcaaaagttgaaaaccttaatgcagctttgaatgaattcaaaaatggcaaaaataaggatttcagcaaggtaacgcaagcaaaaagcgaccttgaaaattccgttaaagatgtgatcatcaatcaaaagataacggataaagttgacaatctcaatcaagcggtatcaatggctaaagcaacgggtgatttcagtagggtagagcaagcgttagccgatctcaaaaacttctcaaaggagcaattggctcaacaaactcaaaaaaatgaaagtttcaatgttggaaaaaaatctgaaatatatcaatccgttaagaatggtgtgaacggaaccctagtcggtaatgggttatctggaatagaggccacagctctcgccaaaaatttttcggatatcaagaaagaattgaatgagaaatttaaaaatttcaataacaataacaataatggactcgaaaacgaacccatttatgctaaagttaataaaaagaaaacaggacaagtagctagccctgaagaacccatttacgctcaagttgctaaaaaggtgaatgcaaaaattgaccgactcaatcaagcagcaagtggtttgggtggtgtagggcaagcgggcttccctttgaaaaggcatgataaagttgatgatctcagtaaggtagggcgatcagttagccctgaacccatttatgctacgattgatgatctcggcggacctttccctttgaaaaggcatgataaagttgatgatctcagtaaggtagggcgatcagttagccctgaacccatttatgctacgattgatgatctcggcggacctttccctttgaaaaggcatgataaagttgatgatctcagtaaggtagggcgatcagttagccctgaacccatttatgctacgattgatgatctcggcggacctttccctttgaaaaggcatgataaagttgatgatctcagtaaggtagggctttcaaggaatcaagaattggctcagaaaattgacaatctcagtcaagcggtatcagaagctaaagcaggtttttttagcaatctagagcaaacgatagacaagctcaaagattctacaaaatacaattctgtgaatctatgggttgaaagtgcaaaaaaagtgcctgctagtttgtcagcgaaactagacaattacgctactaacagccacacacgcattaatagcaatatccaaaatggagcaatcaatgaaaaagcgaccggtatgctaacgcaaaaaaaccctgagtggctcaagctcgtgaatgataagatagttgcgcataatgtgggaagcgttcctttgtcagagtatgataaaattggcttcaaccagaagaatatgaaagattattctgattcgttcaagttttccaccaagttgaacaatgctgtaaaagacgttaagtctagctttacgcaatttttagccaatgcattttctacaggatattactccttggcgagggaaaatgcggagcatggaatcaaaaatgttaatacaaaaggtggtttccaaaaatcttaa

SEQ ID NO: 7 - DNA, 576 bp, synthetic construct
  source 1..576: mol_type = other DNA
atgttagaaaaatcttttttaaaaagcaagcaattatttttatgcggactgggtgttttgatgctgcaggcttgcacttgcccaaacacttcacaaaggaattctttcttgcaagatgtgccttattggatgttgcaaaatcgcagtgagtatatcacgcaaggggtggatagctcgcacattgtagatggtaagaaaactgaagagatagaaaaaatcgctaccaaaagagcgacaataagagtggcacaaaatattgtgcataaacttaaagaagcttacctttccaaaaccaatcgcatcaagcaaaagatcactaatgaaatgtttatccaaatgacacagcccatttatgacagcttgatgaatgtggatcgtttagggatttatatcaatcctaacaatgaggaagtgtttgcgttagtgcgcgcgcgtggttttgataaggacgctttgagcgaagggttgcataaaatgtccttagacaatcaagcggtgagtatccttgtggctaaagtggaagaaatctttaaagattctgtcaattacggagatgttaaagtccctatagccatg

SEQ ID NO: 8 - DNA, 900 bp, synthetic construct
  source 1..900: mol_type = other DNA
atgagaaaaacgatttcagcgttgtttttatcagcgtgcatagggttatcgtctgtttatgcagataacgctttgattttgcaaaccgattttagtctaaaagatggggccgtctcggcgatgaaaggcgtcgctttcagcgttgattcccatcttaaaatctttgatttaacgcacgaaatccccccgtataacatctgggaaggcgcttaccgcttgtatcagaccgccagttattggccaaaaggttcggtatttgtgagcgtagttgatccgggcgtaggcactaagcgtaaatcggtggtactaaaaactaaaaacggccagtatttcgtctcgccggataacggcacgctgactttggtggcacaaactttggggattgatagcgtgcgtgaaattgatgaaaaagctaaccgcttgaaaggttctgaaaaatcctatactttccatggtcgtgatgtgtatgcttacaccggtgcacgcttggcttctggggcgatcacattcgagcaggtcgggccagagcttcccccaaaagtcgttgaaattccttaccaaaaagcgaaagccacaaaaggggaagtgaaaggtaatatcccgattttggatattcaatatggcaatgtttggagcaacatcagcgataaattactcaatcaagcaaaaatcaaactcaatgacacgctgtgtgtaacgatttttaaaggttctaagaaacaatacgaagggaaaatgccgtatgtcgcaagctttggcgatgtgccagaaggccagccgttagtttatttaaacagcttgttaaatgtttccgtggcgctgaatagggataatttcgcgcaaaaatatcaaatcaaatccggtgctgactggaatattgatataaagaagtgcgctaag

SEQ ID NO: 9 - DNA, 726 bp, synthetic construct
  source 1..726: mol_type = other DNA
atgagcgaacaacgacaagaatctttacaaaataaccctaatttgagtaaaaaagatgtcaaaatcgtagaaaagattttgagtaagaacgacattaaagccgctgaaatgaaagaacgctatttaaaagaagggctgtatgtgttgaatttcatgagttctcccggtagcggtaaaaccacgatgctagaaaatctagcggattttaaagactttaagttttgcgtggtagagggcgatttgcaaaccaacagagatgcggacagattgcgtaaaaaaggcgtgagtgcgcaccagatcaccaccggcgaagcatgccatttggaagcgagcatgattgaaggggcgtttgatttattaaaagatgagggagcgttagaaaaaagcgattttttaatcattgaaaacgtggggaatttggtttgcccctcaagctataatctaggagcggcgatgaatatcgttttactctccgttccagagggcgatgataaggtgctaaaataccctacgatgttcatgtgcgcggatgcggtgattatcagtaaagcggatatggttgaggtgtttaatttcagggtttctcaagtcaaagaagacatgcaaaaattaaagcctgaagcgcctatttttttaatgagctctaaagaccctaaaagtttggaagattttaaaaatttccttctagaaaaaaagcgtgaaaattaccagtccacgcattcgttt

SEQ ID NO: 10 - DNA, 537 bp, synthetic construct
  source 1..537: mol_type = other DNA
atgaagagatcttctgtatttagtttcttggtagcttttttattggtagctggctgtagtcataaaatggataataagactgtggccggcgatgtgagtgctaaaacggttcagactgcacctgttactacagaaccagctccagagaaagaagagcctaaacaagagccagctccagtggttgaagaaaaaccggctgttgagagcgggactatcatcgcttctatttattttgattttgacaagtatgaaatcaaagaatccgatcaagagactttagatgagatcgtgcaaaaagctaaagaaaaccacatgcaagtgcttttggaaggcaataccgatgaatttggctctagcgaatacaaccaagcgcttggcgttaaaaggactttgagcgtgaaaaacgctttagtcattaaaggggtagaaaaagatatgatcaaaaccatcagttttggtgaaaccaaacccaaatgcgcccaaaaaactagagagtgttataaagaaaacagaagagtggatgtcaaattaatgaag

SEQ ID NO: 11 - DNA, 1923 bp, synthetic construct
  source 1..1923: mol_type = other DNA
atgaaaaaaacgaaaaaaacgattctgctttctctaactctcgcggcgtcattgctccatgctgaagacaacggcgtttttttaagcgtgggttatcaaatcggtgaagcggttcaaaaagtgaaaaacgccgacaaggtgcaaaaactttcagacacttatgaacaattaagccggcttttaaccaacgataatggcacaaactcaaagacaagcgcgcaagcgatcaaccaagcggttaataatttgaacgaacgcgcaaaaactttagccggtgggacaaccaattcccctgcctatcaagccacgcttttagcgttgagatcggtgttagggctatggaatagcatgggttatgcggtcatatgcggaggttataccaaaagtccaggcgaaaacaatcaaaaagatttccactacaccgatgagaatggcaatggcactacaatcaattgcggtgggagcacaaatagtaatggcactcatagttctagtggcacaaatacattaaaagcagacaaaaatgtttctctatctattgagcaatatgaaaaaatccatgaagcttatcagattctttcaaaagctttaaaacaagccgggcttgctcctttaaatagcaaaggggaaaagttagaagcgcatgtaaccacatcaaaaccagaaaataatagtcaaactaaaacgacaacttctgttattgatacgactaatgatgcgcaaaatcttttgactcaagcgcaaacgattgtcaatacccttaaagattattgccccatgttgatagcgaaatctagtagtgaaagtagtggcgcagctactacaaacgccccttcatggcaaacagccggtggcggcaaaaattcatgtgcgacttttggtgcggagtttagtgccgcttcagacatgattaataatgcgcaaaaaatcgttcaagaaacccaacaactcagcgccaaccaaccaaaaaatatcacacaaccccataatctcaaccttaacacccctagcagtcttacggctttagctcaaaaaatgctcaaaaatgcgcaatctcaagcagaaattttaaaactagccaatcaagtggagagcgattttaacaaactttcttcaggccatcttaaagactacatagggaaatgcgatgcgagcgctataagcagtgcgaatatgacaatgcaaaatcaaaagaacaattgggggaacgggtgtgctggcgtggaagaaactctgtcttcattaaaaacaagtgccgctgattttaacaaccaaacgccacaaatcaatcaagcgcaaaacctagccaacacccttattcaagaacttggcaacaacccttttaggaatatgggcatgatcgcttcttcaaccacgaataacggcgccttgaatggccttggggtgcaagtgggttataagcaattttttggggaaaagaaaagatgggggttaaggtattatggtttctttgattacaaccacgcctatatcaaatccaatttctttaactcggcttctgatgtgtggacttatggggtgggcagcgatttattgtttaatttcatcaatgataaaaacaccaactttttaggcaagaataacaagatttcagtgggattttttggaggtatcgccttagcagggacttcatggcttaattctcaattcgtgaatttaaaaaccatcagcaatgtttatagcgctaaagtgaatacggctaacttccaatttttattcaatttgggcttgagaaccaatctcgctagacctaagaaaaaagatagtcatcatgcggctcaacatggcatggaattgggcgtgaaaatccctaccattaacacgaattattattcttttctagacactaaactagaatatcgaaggctttatagcgtgtatctcaattatgtgtttgcctat